(19) United States
(12) Patent Application Publication
(10) Pub. No.: US 2025/0261909 A1
(43) Pub. Date: Aug. 21, 2025

(54) METHOD AND DEVICE TO PREDICT EXERCISE PEAK VO2, CARDIOVASCULAR OUTCOMES AND FUTURE DEATH USING ECG DEEP LEARNING MODELS

(71) Applicants: The General Hospital Corporation, Boston, MA (US); The Broad Institute, Inc., Cambridge, MA (US)

(72) Inventors: Patrick Ellinor, Boston, MA (US); Steven Lubitz, Boston, MA (US); Samuel Friedman, Cambridge, MA (US); Puneet Batra, Cambridge, MA (US); Nathaniel Diamant, Cambridge, MA (US); Shaan Khurshid, Boston, MA (US); James Sawalla Guseh, Boston, MA (US)

(73) Assignees: The General Hospital Corporation, Boston, MA (US); The Broad Institute, Inc., Cambridge, MA (US)

(21) Appl. No.: 19/197,587

(22) Filed: May 2, 2025

Related U.S. Application Data
(63) Continuation of application No. PCT/US2023/036750, filed on Nov. 3, 2023.
(60) Provisional application No. 63/422,840, filed on Nov. 4, 2022.

(51) Int. Cl. A61B 5/00; A61B 5/349; G16H 50/30

(52) U.S. Cl. CPC A61B 5/7267; A61B 5/349; A61B 5/4884; G16H 50/30

(57) ABSTRACT

A computer-implemented method to determine peak oxygen consumption (\dot{V}O_{2 PEAK}) from electrocardiogram (ECG) waveform data includes the steps of recording, by at least one first computing device, an electrocardiogram (ECG) waveform data of a subject; transmitting, by the at least one first computing device, the ECG waveform data, to at least one second computing device communicatively coupled to the at least one first computing device; determining, by the at least one second computing device, a \dot{V}O_{2 PEAK} of the subject using a convolutional neural network; and transferring, by the at least one second computing device, the \dot{V}O_{2 PEAK} to the first computing device associated or a device associated with the subject.

### CROSS-REFERENCE TO RELATED APPLICATIONS

This application claims the benefit of and priority to International Application No. PCT/US2023/036750, filed on Nov. 3, 2023, which claims the benefit of and priority to U.S. Provisional Patent Application No. 63/422,840, filed on Nov. 4, 2022, the contents of which are incorporated by reference herein in their entireties.

### STATEMENT REGARDING FEDERALLY SPONSORED RESEARCH

This invention was made with government support under Grant Nos. HL 139731 and HL 157635, awarded by the National Institutes of Health. The government has certain rights in the invention.

### TECHNICAL FIELD

The subject matter disclosed herein relates to utilizing electrocardiogram data to predict oxygen consumption (\dot{V}O_{2 PEAK}), potential cardiovascular outcomes, including diseases or disease etiologies, and death. Particular examples relate to providing a system, a computer-implemented method, and a device to utilize electrocardiogram (ECG) waveform data of a subject to determine \dot{V}O_{2 PEAK} with deep learning models.

### BACKGROUND

Oxygen consumption at peak exercise (\dot{V}O_{2 PEAK}) is an integrative marker of cardiorespiratory fitness (CRF) and is among the most powerful prognostic metrics of human health. Across diverse populations, \dot{V}O_{2 PEAK} is a robust and independent predictor of overall mortality, multiple adverse cardiovascular outcomes, and a wide range of other important conditions including malignancy, diabetes, dementia, and many others. However, accurate assessment of \dot{V}O_{2 PEAK} requires dedicated cardiopulmonary exercise testing (CPET), which is resource-intensive, dependent on interpretation expertise, and not universally available.

As a result, despite its broad utility, quantified CRF is currently not routinely assessed in clinical practice, with the most common clinical use of \dot{V}O_{2 PEAK} occurring in the relatively narrow area of clinical assessment of patients with advanced heart failure to guide evaluation for advanced therapies including heart transplantation. However, its predictive value extends into disease-free populations. Deep learning models utilizing resting 12-lead electrocardiograms (ECGs) can extract rich elements of cardiac structure, function, and even future disease susceptibility. However, a need exists for a method, system, and device to estimate \dot{V}O_{2 PEAK} using resting 12-lead ECG, thereby providing automated, instantaneous, and widespread access to this important metric. Specifically, a method, system, and device, which preferably can be a device worn by the user, are needed that can utilize ECG waveform data to predict a subject's \dot{V}O_{2 PEAK} by training deep learning models.

Citation or identification of any document in this application is not an admission that such a document is available as prior art to the present invention.

### SUMMARY

In certain example embodiments, a computer-implemented method to determine peak oxygen consumption (\dot{V}O_{2 PEAK}) from electrocardiogram (ECG) waveform data comprises recording, by at least one first computing device, an electrocardiogram (ECG) waveform data of a subject; transmitting, by the at least one first computing device, the ECG waveform data, to at least one second computing device communicatively coupled to the at least one first computing device; determining, by the at least one second computing device, a \dot{V}O_{2 PEAK} of the subject using a convolutional neural network; and transferring, by the at least one second computing device, the \dot{V}O_{2 PEAK} to the first computing device associated or a device associated with the subject.

In another embodiment, a computer-implemented method to detect one or more diseases or disease etiologies, comprises by at least one computing device: recording ECG waveform data of a subject; generating an estimate of \dot{V}O_{2 PEAK} of the subject using a convolutional neural network; and displaying the estimate of \dot{V}O_{2 PEAK} on a user interface of the at least one computing device.

In a further embodiment, a convolutional neural network generates an at least 320-dimensional embedding of resting 12-lead ECGs.

The convolutional neural network may be a pre-trained convolutional neural network. The convolutional neural network is trained using a training set comprising data from subjects that had at least one ECG within at least 1 year of a \dot{V}O_{2 PEAK} measurement determined using cardiopulmonary exercise testing (CPET).

In one embodiment, training the convolutional neural network further comprises use of a validation data set to which a penalization method has been applied. The penalization method may be an Elastic Net penalization method.

In one embodiment, the computer-implemented methods further comprise generating an estimate of cardiorespiratory fitness (CRF) based on the estimate of a peak oxygen consumption \dot{V}O_{2 PEAK}.

In a further embodiment, the computer-implemented methods further comprise generating an assessment of adverse cardiovascular risk for the subject based on the estimated \dot{V}O_{2 PEAK}.

The assessment of adverse cardiovascular risk is determined using a Cox proportional hazards model with the estimated \dot{V}O_{2 PEAK} as an exposure of interest.

In one embodiment, the adverse cardiovascular event is atrial fibrillation, myocardial infarction, heart failure, or all-cause mortality.

In another embodiment, a device to determine peak oxygen consumption (\dot{V}O_{2 PEAK}) from electrocardiogram (ECG) waveform data comprises: electrocardiogram leads for obtaining ECG waveform data from a subject; a user interface; a storage device; and a processor communicatively coupled to the storage device, wherein the processor executes application code instructions that are stored in the storage device to cause the device to: record an ECG waveform data of a subject; determine a \dot{V}O_{2 PEAK} of the subject using a convolutional neural network; and display the \dot{V}O_{2 PEAK} via a user interface.

In one embodiment, a convolutional neural network generates an at least 320-dimensional embedding of resting 12-lead ECGs.

In a further embodiment, the convolutional neural network is a pre-trained convolutional neural network.

In another embodiment, training the convolutional neural network further comprises use of a validation data set to which a penalization method has been applied. The penalization method is an Elastic Net penalization method.

In one embodiment, the device further comprises application code instructions that cause the device to generate an estimate of cardiorespiratory fitness (CRF) based on the estimate of a peak oxygen consumption \dot{V}O_{2 PEAK}.

In another embodiment, the device further comprises application code instructions that cause the device to generate an assessment of adverse cardiovascular risk for the subject based on the estimated \dot{V}O_{2 PEAK}.

In an embodiment, the assessment of adverse cardiovascular risk is determined using a Cox proportional hazards model with the estimated \dot{V}O_{2 PEAK} as an exposure of interest.

In a further embodiment, the adverse cardiovascular event is atrial fibrillation, myocardial infarction, heart failure, or all-cause mortality.

In another embodiment, the device is a wearable device.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 is an overview of the study design. First, Applicants analyzed 2,339 individuals who underwent cardiopulmonary exercise testing (CPET) at Massachusetts General Hospital (MGH, top panel). Applicants fit models in 1,891 individuals, who were in turn divided into training and validation sets. The best performing models were then evaluated in a separate sample of 448 individuals not included in model training. Second, Applicants used the Deep ECG-\dot{V}O₂ model to infer \dot{V}O_{2 PEAK} in 84,718 individuals in a Mass General Brigham (MGB)-based ambulatory cohort with a 12-lead electrocardiogram (ECG) performed within three years prior to start of follow-up (bottom panel). Applicants then assessed whether predicted \dot{V}O_{2 PEAK} was associated with longitudinal disease incidence.

FIG. 2 shows a distribution of cardiopulmonary exercise test derived \dot{V}O_{2 PEAK} in the full analysis set (n=2,339) and additionally for women (n=876) and men (n=1,463).

FIG. 3 shows a histogram depicting the number of days between the 12-lead ECG used for \dot{V}O_{2 PEAK} prediction, to the CPET from which \dot{V}O_{2 PEAK} was obtained. Negative values represent number of days by which the ECG preceded CPET, and positive values represent the number of days by which ECG came after CPET. The median time from ECG to CPET was 7 days (quartile-1: 27 days, quartile-3: 0 days). A total of 130 outlying observations are not depicted for graphical purposes.

FIG. 4A shows an absolute error, and FIG. 4B shows a relative error comparing Deep ECG-\dot{V}O₂ estimated \dot{V}O_{2 PEAK} versus true \dot{V}O_{2 PEAK} in the model test set (n=448). Error is shown per increasing decile of true \dot{V}O_{2 PEAK}. Y-axis values reflect true minus estimated \dot{V}O_{2 PEAK}, with negative values reflecting model overestimations and positive values reflecting model underestimations.

FIG. 5 shows a distribution of Deep ECG-\dot{V}O₂ predicted cardiopulmonary exercise test-derived \dot{V}O_{2 PEAK} in the disease analysis set (n=84,718). Twenty-one (0.02%) outlying values are not depicted for graphical purposes.

FIG. 6 shows a plot of the adjusted cumulative risk of atrial fibrillation, myocardial infarction, heart failure, and all-cause death for women, stratified by Deep ECG-\dot{V}O₂ predicted \dot{V}O_{2 PEAK} below 14 mL/kg/min (gray) versus above at or above 14 mL/kg/min (black). The threshold 14 mL/kg/min is used clinically to define substantially impaired cardiorespiratory fitness. Curves are derived from stratified Cox models with each respective disease as the outcome and predicted \dot{V}O_{2 PEAK} below 14 mL/kg/min as a stratification variable. Age and body mass index were included as covariates, with the sex-specific mean value assumed.

FIG. 7 shows plots of the adjusted cumulative risk of atrial fibrillation, myocardial infarction, heart failure, and all-cause death for men, stratified by Deep ECG-\dot{V}O₂ predicted \dot{V}O_{2 PEAK} below 14 mL/kg/min (gray) versus above at or above 14 mL/kg/min (black). The threshold 14 mL/kg/min is used clinically to define substantially impaired cardiorespiratory fitness. Curves are derived from stratified Cox models with each respective disease as the outcome and predicted \dot{V}O_{2 PEAK} below 14 mL/kg/min as a stratification variable. Age and body mass index were included as covariates, with the sex-specific mean value assumed.

FIG. 8 shows plots of the cumulative risk of atrial fibrillation, myocardial infarction, heart failure, and all-cause death, stratified by Deep ECG-\dot{V}O₂ predicted \dot{V}O_{2 PEAK} at the 25^{th} percentile. The sample-based 25^{th} percentile of estimated \dot{V}O_{2 PEAK} is approximately 24.2 mL/kg/min. The number at risk within each stratum over time is depicted below each plot.

FIG. 9 shows plots of the adjusted cumulative risk of atrial fibrillation, myocardial infarction, heart failure, and all-cause death for women, stratified by Deep ECG-\dot{V}O₂ predicted \dot{V}O_{2 PEAK} below the 25^{th} percentile (gray) versus at or above the 25^{th} percentile (black). Curves are derived from stratified Cox models with each respective disease as the outcome and predicted \dot{V}O_{2 PEAK} below the 25^{th} percentile as a stratification variable. The sample-based 25^{th} percentile of estimated \dot{V}O_{2 PEAK} was approximately 24.2 mL/kg/min. Age and body mass index were included as covariates, with the sex-specific mean value assumed.

FIG. 10 shows plots of the adjusted cumulative risk of atrial fibrillation, myocardial infarction, heart failure, and all-cause death for men, stratified by Deep ECG-\dot{V}O₂ predicted \dot{V}O_{2 PEAK} below the 25^{th} percentile (gray) versus at or above the 25^{th} percentile (black). Curves are derived from stratified Cox models with each respective disease as the outcome and predicted \dot{V}O_{2 PEAK} below the 25^{th} percentile as a stratification variable. The sample-based 25^{th} percentile of estimated \dot{V}O_{2 PEAK} was approximately 24.2 mL/kg/min. Age and body mass index were included as covariates, with the sex-specific mean value assumed.

FIGS. 11A and 11B show correlation and agreement between model estimated \dot{V}O_{2 PEAK} versus true \dot{V}O_{2 PEAK}. FIG. 11A depicts correlation plots comparing resting 12-lead ECG estimated \dot{V}O_{2 PEAK} (y-axis). The Basic model contains age, sex, body mass index, and CPET modality. The Basic+ECG Parameters model contains age, sex, body mass index, CPET modality, and tabular ECG measurements. Deep ECG-\dot{V}O₂ is a model containing age, sex, body mass index, CPET modality, and deep learned ECG embeddings. The Pearson correlation (r) and Mean Absolute Error (MAE), with associated 95% confidence intervals, are depicted on the bottom right of each plot. The diagonal line represents perfect correlation. FIG. 11B depicts Bland-Altman plots, which show agreement between resting 12-lead ECG estimated \dot{V}O_{2 PEAK} and true cardiopulmonary exercise test-derived \dot{V}O_{2 PEAK}. On each plot, the x-axis depicts the mean of paired values for an individual, and the y-axis plots the difference (i.e., true \dot{V}O_{2 PEAK} minus estimated \dot{V}O_{2 PEAK}). Therefore, positive y-axis values demonstrate underestimations by the model, and vice versa. The central solid horizontal line depicts the overall mean difference, and the hashed lines depict the estimated 95% limits of agreement. An accurate model has y-axis values close to zero, and an unbiased model shows no systematic pattern along the x-axis (i.e., random scatter). The values corresponding to the 95% limits of agreement are shown on each plot, along with the actual proportion of points falling within those limits.

FIGS. 12A and 12B show a cumulative risk of disease stratified by Deep ECG-\dot{V}O₂ estimated \dot{V}O_{2 PEAK}. Plots depict the cumulative risk of atrial fibrillation (left), myocardial infarction (middle left), heart failure (middle right), and all-cause death (right). FIG. 12A depicts cumulative risk stratified by Deep ECG-\dot{V}O₂ estimated \dot{V}O_{2 PEAK} below 14 mL/kg/min (gray) versus above at or above 14 mL/kg/min (black). The threshold 15 mL/kg/min is commonly used to define severely impaired cardiorespiratory fitness. FIG. 12B depicts cumulative risk stratified by sample-level quartile of Deep ECG-\dot{V}O₂ estimated \dot{V}O_{2 PEAK}, approximately corresponding to: Quartile-1<24.2; Quartile-2 24.2-30.1; Quartile-3 30.2-36.2; Quartile-4>35.2. The number at risk within each stratum over time is depicted below each plot.

FIG. 13 shows median waveforms stratified by Deep ECG-\dot{V}O₂ estimated \dot{V}O_{2 PEAK}. Depicted are median waveforms taken from individuals in the test set not used for model training (n=448). The black waveforms represent the median waveforms observed for individuals at or above the 90^{th} percentile of Deep ECG-\dot{V}O₂ estimated \dot{V}O_{2 PEAK} (i.e., >48 mL/kg/min), while gray waveforms represent the median waveforms observed for individuals below the 10^{th} percentile of Deep ECG-\dot{V}O₂ estimated \dot{V}O_{2 PEAK} (i.e., <16 mL/kg/min).

FIG. 14 shows a block diagram depicting a system to determine peak oxygen consumption (\dot{V}O_{2 PEAK}) from electrocardiogram (ECG) waveform data and perform machine learning on ECG waveform data of a subject.

FIG. 15 shows a block flow diagram illustrating methods to determine peak oxygen consumption (\dot{V}O_{2 PEAK}) from electrocardiogram (ECG) waveform data, in accordance with certain examples of the technology disclosed herein.

FIG. 16 shows a block diagram depicting a computing machine and modules, in accordance with certain examples of the technology disclosed herein.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a,” “an,” and “the” include both singular and plural referents unless the context clearly dictates otherwise.

The term “optional” or “optionally” means that the subsequent described event, circumstance, or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar as such variations are appropriate to perform in the disclosure herein. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” and “an example embodiment” means that a particular feature, structure, or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

In one aspect, embodiments disclosed herein provide methods to predict a subject's peak oxygen consumption (\dot{V}O_{2 PEAK}) using the subject's ECG waveform data and a deep learning model, thereby providing information on the subject's potential cardiovascular outcomes and cardiorespiratory fitness (CRF). The use of Deep ECG-\dot{V}O₂ may be particularly useful for the detection of individuals with impaired CRY, given favorable sensitivity and case reclassification when compared to baseline models. The current standard for determining a subject's CRF is to conduct a cardiopulmonary exercise test (CPET), which typically involves bulky and expensive exercise equipment in a CPET lab, which is not available at every medical facility. Furthermore, it is difficult to train machine learning approaches with data such as an ECG waveform, as there is no expansive, pre-labeled training set. The potential clinical applications of this approach are broad, as accurate \dot{V}O_{2 PEAK} estimation could have powerful implications for varied clinical populations where CRF assessment is prognostically useful but access to a dedicated CPET lab is limited, or exercise is not feasible. The disclosed methods, systems, and devices for predicting \dot{V}O_{2 PEAK}, based on using standard 12-lead ECG waveform data, provide an important advance, as the disclosure herein demonstrates that \dot{V}O_{2 PEAK} is a measure of CRF and can be correlated further to adverse cardiovascular events. The solutions provided herein are therefore non-conventional and technology based.

In certain example embodiments, a computer-implemented method to determine peak oxygen consumption (\dot{V}O_{2 PEAK}) from electrocardiogram (ECG) waveform data comprises recording, by at least one first computing device, an electrocardiogram (ECG) waveform data of a subject; transmitting, by the at least one first computing device, the ECG waveform data, to at least one second computing device communicatively coupled to the at least one first computing device; determining, by the at least one second computing device, a \dot{V}O_{2 PEAK} of the subject using a convolutional neural network; and transferring, by the at least one second computing device, the \dot{V}O_{2 PEAK} to the first computing device associated or a device associated with the subject.

In another embodiment, a computer-implemented method to detect one or more diseases or disease etiologies, comprises by at least one computing device: recording ECG waveform data of a subject; generating an estimate of \dot{V}O_{2 PEAK} of the subject based using a convolutional neural network; and displaying the estimate of \dot{V}O_{2 PEAK} on a user interface of the at least one computing device.

In another embodiment, a device to determine peak oxygen consumption (\dot{V}O_{2 PEAK}) from electrocardiogram (ECG) waveform data comprises: electrocardiogram leads for obtaining ECG waveform data from a subject; a user interface; a storage device; and a processor communicatively coupled to the storage device, wherein the processor executes application code instructions that are stored in the storage device to cause the device to: record an ECG waveform data of a subject; determine a \dot{V}O_{2 PEAK} of the subject using a convolutional neural network; and display the \dot{V}O_{2 PEAK} via a user interface.

The embodiments described herein include computer-implemented methods, computer program products, and devices to use ECG waveform data to predict a subject's \dot{V}O_{2 PEAK} and predict cardiovascular outcomes, including future death using deep learning models.

Standard techniques related to making and using aspects of the invention may or may not be described in detail herein. Various aspects of computing systems and specific computer programs to implement the various technical features described herein are well known.

### Example System Architectures

Turning now to the drawings, in which like numerals represent like (but not necessarily identical) elements throughout the figures, example embodiments are described in detail.

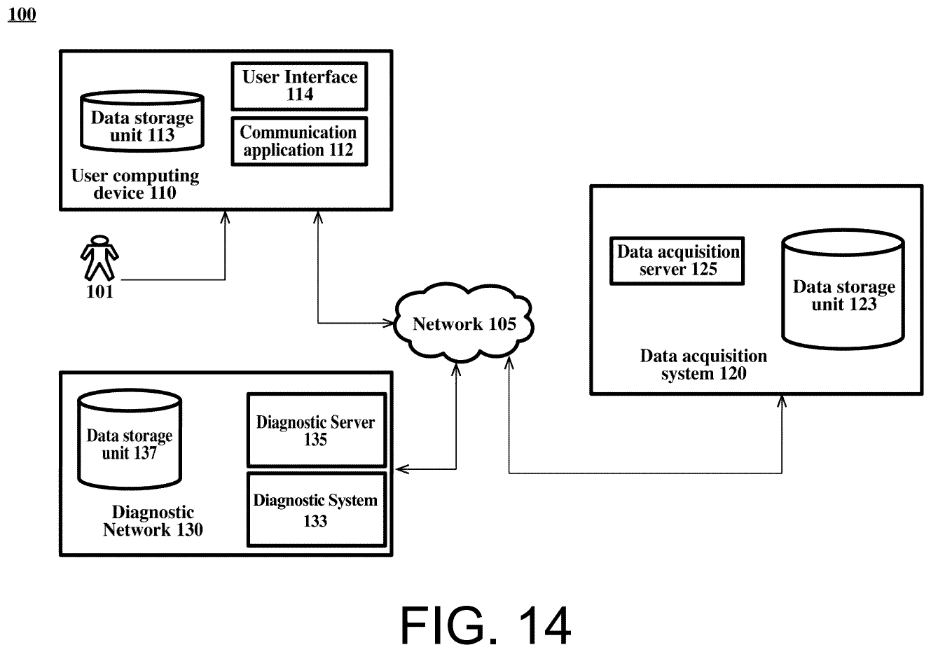

FIG. 14 is a block diagram depicting a system 100 to determine peak oxygen consumption (\dot{V}O_{2 PEAK}) from electrocardiogram (ECG) waveform data and perform machine learning on ECG waveform data of a subject. In one example embodiment, a user 101 associated with a user computing device 110 installs an application and/or makes a feature selection to obtain the benefits of the techniques described herein.

As depicted in FIG. 14, the system 100 includes networked computing devices/systems, depicted as a user computing device 110, a data acquisition system 120, and a diagnostic network 130, that are configured to communicate with one another via one or more networks 105 or via any suitable communication technology.

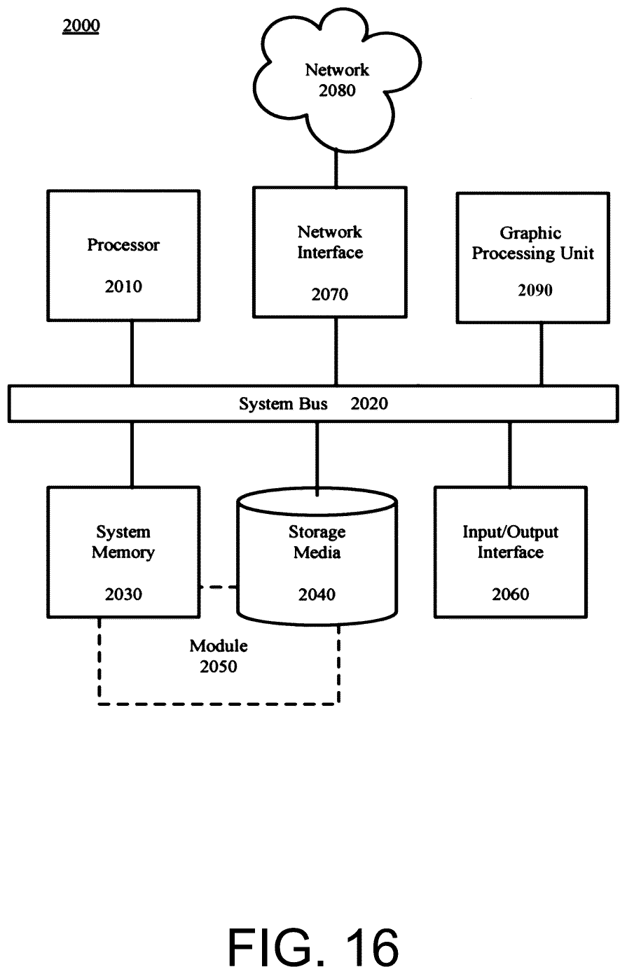

Each network 105 includes a wired or wireless telecommunication means by which network computing devices/systems (including the network computing devices/systems described herein) can exchange data. For example, each network 105 can include any of those described herein, such as the network 2080 described in FIG. 16, any combination thereof, or any other appropriate architecture or system that facilitates the communication of signals and data. Throughout the discussion of example embodiments, it should be understood that the terms “data” and “information” are used interchangeably herein to refer to text, images, audio, video, or any other form of information that can exist in a computer-based environment. The communication technology utilized by the network computing devices/systems may be similar to the network 105 or may be an alternative communication technology.

Each network computing device/system includes a computing device having a communication module capable of transmitting and receiving data over the network 105 or a similar network. For example, each of the user computing device 110, the data acquisition system 120, and the diagnostic network 130 can include any computing machine 2000 described herein and found in FIG. 16 or any other wired or wireless, processor-driven device. In the example embodiment depicted in FIG. 14, the user computing device 110, the data acquisition system 120, and the diagnostic network 130 are operated by the user 101, data acquisition system operators, and diagnostic network operators, respectively.

The user computing device 110 includes a user interface 114. The user interface 114 may be used to display a graphical user interface and other information to the user 101 to allow the user 101 to interact with the data acquisition system 120, the diagnostic network 130, and others. The user interface 114 receives user input for data acquisition and/or machine learning and displays results to the user 101. In another example embodiment, the user interface 114 may be provided with a graphical user interface by the data acquisition system 120 and/or the diagnostic network 130. The user interface 114 may be accessed by the processor of the user computing device 110. The user interface 114 may display a webpage associated with the data acquisition system 120 and/or the diagnostic network 130. The user interface 114 may be used to provide input, configuration data, and other display direction by the webpage of the data acquisition system 120 and/or the diagnostic network 130. In another example embodiment, the user interface 114 may be managed by the data acquisition system 120, the diagnostic network 130, or others. In another example embodiment, the user interface 114 may be managed by the user computing device 110 and may be prepared and displayed to the user 101 based on the operations of the user computing device 110.

In one aspect, the user computing device 110 may be a portable device that may comprise a 12-lead ECG monitor to obtain ECG waveform data from a subject wearing or carrying the portable device. The user computing device 110 may further comprise a storage device and a processor communicatively coupled to the storage device. The processor may further comprise a machine learning/AI engine to perform the machine learning processing disclosed herein on the user computing device 110. The user computing device 110, therefore, may be configured to serve as a device to record ECG waveform data from the subject (i.e., an ECG waveform acquisition device), to process the acquired ECG waveform data using a machine learning model, and to generate a diagnostic output via the machine learning analysis of the ECG waveforms.

The user 101 can use a communication application 112 on the user computing device 110, which may be, for example, a web browser application or a stand-alone application, to view, download, upload, or otherwise access documents or web pages through the user interface 114 via the network 105. The user computing device 110 can interact with the web servers or other computing devices connected to the network, including a data acquisition server 125 of the data acquisition system 120 and a diagnostic server 135 of the diagnostic network 130. In another example embodiment, the user computing device 110 communicates with devices in the data acquisition system 120 and/or the diagnostic network 130 via any other suitable technology, including the example computing system described below.

The user computing device 110 also includes a data storage unit 113 accessible by the user interface 114, the communication application 112, or other applications. The example data storage unit 113 can include one or more tangible computer-readable storage devices. The data storage unit 113 can be stored on the user computing device 110 or can be logically coupled to the user computing device 110. For example, the data storage unit 113 can include on-board flash memory and/or one or more removable memory accounts or removable flash memory. In another example embodiments, the data storage unit 113 may reside in a cloud-based computing system.

In an example, the data acquisition system 120 comprises a data storage unit 123 and the data acquisition server 125. The data storage unit 123 can include any local or remote data storage structure that is accessible to the data acquisition system 120 and suitable for storing information. The data storage unit 123 can include one or more tangible computer-readable storage devices, or the data storage unit 123 may be a separate system, such as a different physical or virtual machine, or a cloud-based storage service.

In one aspect, the data acquisition server 125 communicates with the user computing device 110 and/or the diagnostic network 130 to transmit requested data. The data may include ECG waveform data.

An example diagnostic network 130 comprises a diagnostic system 133, a diagnostic server 135, and a data storage unit 137. The diagnostic server 135 communicates with the user computing device 110 and/or the data acquisition system 120 to request and receive data. The data may comprise the data types previously described in reference to the data acquisition server 125.

The diagnostic system 133 receives an input of data from the diagnostic server 135. The diagnostic system 133 can comprise one or more functions to implement any of the mentioned training methods to learn peak oxygen consumption (\dot{V}O_{2 PEAK}) from electrocardiogram (ECG) waveform data. In a preferred embodiment, a machine learning model may comprise a convolutional neural network. In one example embodiment, the convolutional neural network may comprise a convolutional auto encoder. In another example embodiment, the convolutional neural network may generate an embedding. Any suitable architecture may be applied to learn peak oxygen consumption (\dot{V}O_{2 PEAK}) from electrocardiogram (ECG) waveform data.

The data storage unit 137 can include any local or remote data storage structure accessible to the diagnostic network 130 suitable for storing information. The data storage unit 137 can include one or more tangible computer-readable storage devices, or the data storage unit 137 may be a separate system, such as a different physical or virtual machine or a cloud-based storage service.

In an alternate embodiment, the functions of either or both of the data acquisition system 120 and the diagnostic network 130 may be performed by the user computing device 110.

It will be appreciated that the network connections shown are examples, and other means of establishing a communications link between the computers and devices can be used. Moreover, those having ordinary skill in the art having the benefit of the present disclosure will appreciate that the user computing device 110, the data acquisition system 120, and the diagnostic network 130 illustrated in FIG. 14 can have any of several other suitable computer system configurations. For example, the user computing device 110, when embodied as a mobile phone or handheld computer, may not include all the components described above.

In example embodiments, the network computing devices and any other computing machines associated with the technology presented herein may be any type of computing machine such as, but not limited to, those discussed in more detail with respect to FIG. 16. Furthermore, any modules associated with any of these computing machines, such as modules described herein, or any other modules (scripts, web content, software, firmware, or hardware) associated with the technology presented herein, may be any of the modules discussed in more detail with respect to FIG. 16. The computing machines discussed herein may communicate with one another as well as other computer machines or communication systems over one or more networks, such as network 105. The network 105 may include any type of data or communications network, including any of the network technology discussed with respect to FIG. 16.

### Example Processes

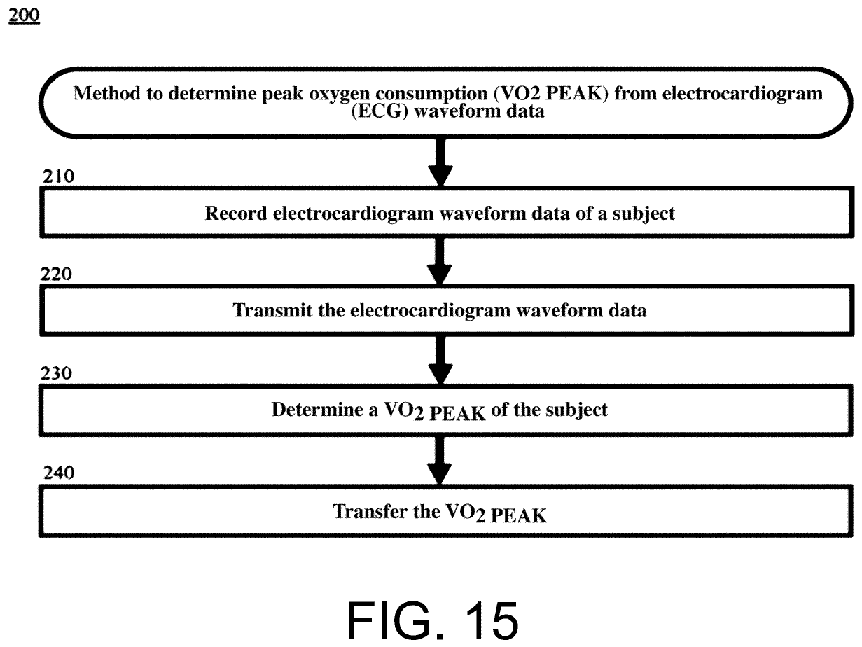

The example method illustrated in FIG. 15 is described hereinafter with respect to the components of the example system 100. The example method also can be performed with other systems and in other architectures including similar elements.

Referring to FIG. 15, and continuing to refer to FIG. 14 for context, a block flow diagram illustrates a method 200 to determine peak oxygen consumption (\dot{V}O_{2 PEAK}) from electrocardiogram (ECG) waveform data, in accordance with certain examples of the technology disclosed herein. In an example embodiment, the methods described herein, including the method 200, may be performed on a single computing device (e.g., at least one computing device), which further comprises the user computing device 110, the diagnostic network 130, the data acquisition system 120, or any combination thereof. In example embodiments, the methods described herein may be performed on a first and second computing device, as also described herein.

In block 210, the user computing device 110 records ECG waveform data. In example embodiments, a separate computing device comprising a recording device first records the ECG waveform data. The ECG waveform data on the separate recording device is transferred from the recording device to the user computing device 110. In example embodiments, the recording device is located on a first computing device (e.g., the user computing device 110) and further transferred to a second computing device. In example embodiments, the recording device is located on a single device. In example embodiments, the recording device is an ECG, an echocardiogram, or a combination thereof.

### ECG Recording Device

In one aspect, the ECG waveform data are recorded with an ECG recording device. ECG recording devices may comprise a standard ECG recording device, an ambulatory ECG recording device, or both. A standard ECG recording device records the electrical activity of a heart (i.e., ECG waveform data) for a short period of time (e.g., approximately 10 seconds). An ambulatory ECG recording device records the electrical activity of a heart for a prolonged period of time (e.g., greater than 10 seconds).

In an example embodiment, the ECG recording device comprises an ambulatory ECG recording device, wherein the electrical activity of the heart is measured for at least 30 seconds, at least 1 minute, at least 5 minutes, at least 30 minutes, at least 1 hour, at least 6 hours, at least 12 hours, at least 1 day, at least 2 days, at least 15 days, at least 1 month, at least 6 months, or at least 1 year. In an example embodiment, the standard or ambulatory ECG device comprises a multi-lead surface monitor, a Holter monitor, an event monitor, a patch monitor, an implantable monitor, or a consumer device (e.g., a smartwatch).

A multi-lead surface monitor typically refers to a standard surface ECG monitor. A standard surface monitor comprises electrodes placed on the surface of a subject. A standard surface ECG monitor may comprise 1- to 12-leads placed at different locations across the subject. The leads capture the differences in electrical activity measured by the electrodes. Electrodes are typically placed at the right arm (RA), left arm (LA), left leg (LL), right leg (RL) and V1-V6 along the chest (precordial). The 12 leads comprise six limb (or extremity) leads and six chest leads. The six limb leads correspond to the RA, LA, LL, and RL electrodes. The six limb leads comprise three bipolar leads (i.e., I, II, and III) and six unipolar leads (i.e., aV_{R}, aV_{L}, and aV_{F}). Lead I corresponds to both electrodes LA and RA, lead II corresponds to both electrodes LL and RA, and lead III corresponds to both electrodes LL and LA. Leads aV_{R}, aV_{L}, and aV_{F} individually correspond to any one of electrodes RA, LA, LL, and RL and are assigned depending on the need during monitoring. The six chest leads correspond to the six chest electrodes. Any two or more electrodes can be used to establish a 1- to 12-lead system. For example, in a 3-lead system, only three electrodes are placed, typically at the RA, LA, and LL locations on the surface of a subject. The three electrodes correspond to the three bipolar leads I, II, and III.

Other ECG recording devices use different combinations of the electrodes to measure the electrical activity of the heart. A Holter monitor is a portable device comprising two or more (e.g., five to seven) leads and continuously records every heartbeat. An event monitor, on the other hand, generally does not attempt to record every heartbeat. In some instances, the event monitor is worn continuously during the course of monitoring. In some instances, the event monitor is worn when symptoms occur. Event monitors worn continuously may comprise automatic symptom detection and only record if a symptom is detected. Event monitors comprising automatic symptom detection may include manual recording as well. Event monitors may be symptom monitors or memory looping monitors. One differentiating feature between symptom and memory looping monitors is memory looping monitors record the electrical activity of the heart for some time before the memory looping monitor is activated.

Patch monitors are self-contained monitors attached directly to a subject. All the components, including the leads, are contained within a single device. Typically, patch monitors comprise an adhesive that attaches directly to a subject. Patch monitors generally record substantially all electrical activity of the heart while monitoring. Some patch monitors can wirelessly transmit recorded electrical activity of the heart. Implantable monitors, or implantable loop monitors, are inserted under the skin of a subject. Typically, implantable monitors record the electrical activity of the heart continuously during monitoring. During operation, the recorded data are periodically transmitted wirelessly to an exterior device for further processing. In some instances, a patch monitor or implantable monitor may use leadless technology.

Consumer devices may comprise any monitor previously mentioned but are designed for personal use. Consumer devices may also comprise wearable mobile devices (e.g., smartwatches). Consumer device monitors may comprise a single device or multiple devices worn on or attached to the body to monitor and record the electrical activity of the heart.

In example embodiments, “leadless” ECG recording devices are used. Leadless ECG recording devices use wireless technology and at least two points of measurement to record the electrical activity of the heart. In example embodiments, leadless ECG recording devices record measurements from two points of the body (e.g., right arm/left arm or right leg/left leg). The two points may be on the surface of a subject (e.g., body) opposite each other where the heart is situated between the two points.

In block 220, the waveform data are transferred (e.g., transmitted) over a network via the transfer engine from the user computing device 110 or the data acquisition system 120 to the diagnostic network 130. The transfer engine comprises any software or hardware individually or in combination, such as those described herein, capable of moving, copying, or otherwise transferring the waveform data, thereby allowing access within the diagnostic network 130. In example embodiments, the waveform data are on a first computing device and transferred to a second computing device. In example embodiments, the waveform data are transferred within the single computing device. In example embodiments, the waveform data are ECG waveform data, echocardiogram waveform data, or a combination thereof.

### ECG Waveform

ECG waveform data may comprise any electrical activity of a subject's heart. This electrical activity can be picked up on the skin as it is transmitted throughout the body. The direction of the ECG waveform data (i.e., direction of a deflection, upward or downward) may vary depending on whether the electrical activity is going towards or away from a lead. The typical characteristic feature of this electrical activity comprises three waves: the P wave, the QRS wave complex, and the T wave.

The P wave typically presents as a small deflection and represents atrial depolarization. The QRS wave complex comprises multiple presentations. For example, if the wave immediately after the P wave deflects downwards, then the first wave in the QRS wave complex is a Q wave. If the wave immediately after the P wave deflects upward, then the first wave in the QRS wave complex is an R wave. The Q wave may comprise various sizes. For example, a small Q wave may correspond to depolarization of the interventricular septum, while big and wide Q waves may correspond to an old myocardial infarction. In general, Q waves correspond to breathing and are small and thin. R waves are typically the largest wave and correspond to the depolarization of the main mass of the ventricles. S waves represent the final depolarization of the ventricles. The T wave represents ventricular repolarization. In an example embodiment, the ECG waveform data may further comprise U waves, which are small waves following T waves.

The typical characteristic features of the electrical activity of a heart may further comprise characteristic features representing the relationship between the three waves. These features may comprise segments or intervals of the wave. A segment is a region between two waves, while an interval is a duration of time between waves and includes a segment. For example, the PR interval includes the PR segment and represents a measure of time between the first deflection of the P wave and the first deflection of the QRS complex. The ST interval includes the ST segment and represents a measure of time between the end of the QRS complex and the end of the T wave. The ST interval characterizes the period of zero potential between ventricular depolarization and repolarization. The QT interval is the time from the start of the Q wave to the end of the T wave and is inversely correlated with heart rate. The QTc-interval is a QT-interval corrected for heart rate. Various formulae to correct QT-intervals are well known in the art and will not be mentioned herein for brevity. Additional features may include the J point, which is the junction between the termination of the QRS complex and the beginning of the ST segment.

Every characteristic feature of ECG waveform data was not mentioned for brevity. One of ordinary skill in the art would immediately recognize features not mentioned herein may be included in ECG waveform data.

In block 230, the diagnostic network 130 or data acquisition system 120 receives input of the ECG waveform data. The diagnostic network 130 may receive the ECG waveform data from the user computing device 110, the data acquisition system 120, or any other suitable source of ECG waveform data via the network 105 to the diagnostic network 130, discussed in more detail in other sections herein. The acquisition engine comprises any software or hardware individually or in combination described herein that is capable or fetching or receiving the ECG waveform data and thereby allowing access to the ECG waveform data by the diagnostic network 130 or the data acquisition system 120. In example embodiments, the second computing device receives the ECG waveform data from the first computing device. In example embodiments, the ECG waveform data are received within a single computing device, wherein the ECG waveform data are recorded by the recording device within the single computing device, and either the diagnostic network 130 or data acquisition system 120 within the single computing device receives the ECG waveform data. The received ECG waveform data are passed to the diagnostic server 135, wherein the waveform data are further processed by the diagnostic system 133.

In block 230, the diagnostic system 133 processes the data of the ECG waveform into output data comprising peak oxygen consumption (\dot{V}O_{2 PEAK}). The diagnostic network 130 or data acquisition system 120 receives input of the ECG waveform data. The diagnostic network 130 may receive the ECG waveform data from the user computing device 110, the data acquisition system 120, or any other suitable source of ECG waveform data via the network 105 to the diagnostic network 130, discussed in more detail in other sections herein. In example embodiments, the second computing device receives the ECG waveform data from the first computing device. In example embodiments, the ECG waveform data are received within a single computing device, wherein the ECG waveform data are recorded by the recording device within the single computing device, and either the diagnostic network 130 or data acquisition system 120 within the single computing device receives the ECG waveform data. The received ECG waveform data are passed to the diagnostic server 135, wherein the ECG waveform data are further processed by the diagnostic system 133. The diagnostic system 133 comprises a machine learning network, as described further herein. The machine learning network analyzes the ECG waveform data, transforming it into output data. The machine learning output may be further processed into user comprehensible information comprising a diagnostic output of \dot{V}O_{2 PEAK} (i.e., generating a diagnostic output). In an example embodiment, the diagnostic output is generated in the diagnostic system 133, which further comprises a module for generating the diagnostic output. In an example embodiment, the machine learning output is passed to the diagnostic server 135, which further comprises a module for generating the diagnostic output. In an example embodiment, the machine learning output is transmitted to the data acquisition system 120 or user computing device 110, wherein the diagnostic output is generated by those devices/systems.

### Peak Oxygen Consumption (\dot{V}O_{2 PEAK})

The highest rate at which oxygen can be consumed during exercise is Peak Oxygen Consumption (\dot{V}O_{2 PEAK}). \dot{V}O_{2 PEAK} represents the limit a body can deliver and extract oxygen in muscles during the metabolic demand of vigorous exercise. In general, \dot{V}O_{2 PEAK} is measured using cardiopulmonary exercise test (CPET), and \dot{V}O_{2 PEAK} increases in accordance with age and maturation as morphological and physiological changes occur. However, \dot{V}O_{2 PEAK} may be further influenced by age-induced neuromuscular deterioration, sarcopenia, and cardiopulmonary decline. \dot{V}O_{2 PEAK} is increasingly used to assess risk and make clinical decisions because it can predict therapeutic response and adverse events. For example, \dot{V}O_{2 PEAK} is associated with coronary artery disease and left heart disease.

The ladder diagrams, scenarios, flowcharts, and block diagrams in the figures and discussed herein illustrate architecture, functionality, and operation of example embodiments and various aspects of systems, methods, and computer program products of the present disclosure. Each block in the flowchart or block diagrams can represent the processing of information and/or transmission of information corresponding to circuitry that can be configured to execute the logical functions of the present techniques. Each block in the flowchart or block diagrams can represent a module, segment, or portion of one or more executable instructions for implementing the specified operation or step. In example embodiments, the functions/acts in a block can occur out of the order shown in the figures, and nothing requires that the operations be performed in the order illustrated. For example, two blocks shown in succession can executed concurrently or essentially concurrently. In another example, blocks can be executed in the reverse order. Furthermore, variations, modifications, substitutions, additions, or reduction in blocks and/or functions may be used with any of the ladder diagrams, scenarios, flow charts, and block diagrams discussed herein, all of which are explicitly contemplated herein.

The ladder diagrams, scenarios, flow charts and block diagrams may be combined with one another, in part or in whole. Coordination will depend upon the required functionality. Each block of the block diagrams and/or flowchart illustration as well as combinations of blocks in the block diagrams and/or flowchart illustrations can be implemented by special purpose hardware-based systems that perform the aforementioned functions/acts or carry out combinations of special purpose hardware and computer instructions. Moreover, a block may represent one or more information transmissions and may correspond to information transmissions among software and/or hardware modules in the same physical device and/or hardware modules in different physical devices.

The present techniques can be implemented as a system, a method, a computer program product, digital electronic circuitry, and/or in computer hardware, firmware, software, or in combinations of them. The system may comprise distinct software modules embodied on a computer-readable storage medium; the modules can include, for example, any or all of the appropriate elements depicted in the block diagrams and/or described herein. By way of example and not limitation, any one, some, or all of the modules/blocks and/or sub-modules/sub-blocks described may be included in the system. The method steps can then be carried out using the distinct software modules and/or sub-modules of the system, as described above, executing on one or more hardware processors.

The computer program product can include a program tangibly embodied in an information carrier (e.g., computer-readable storage medium or media) having computer-readable program instructions thereon for execution by, or to control the operation of, a data processing apparatus (e.g., a processor) to carry out aspects of one or more embodiments of the present disclosure. It will be understood that each block of the flowchart illustrations and/or the block diagrams, and combinations of blocks in the flowchart illustrations and/or the block diagrams, can be implemented by computer-readable program instructions.

The computer-readable program instructions can be performed on a general purpose computing device, a special purpose computing device, or other programmable data processing apparatus to produce a machine. The instructions are executed at least partially by one or more processors that are temporarily configured (e.g., by software) or permanently configured to perform the functions/acts specified in the flowchart and/or block diagram block or blocks. The processors, which may be temporarily, permanently, or partially configured, may comprise processor-implemented modules. The present techniques referred to herein may, in example embodiments, comprise processor-implemented modules. Functions/acts of the processor-implemented modules may be distributed among the one or more processors. Moreover, the functions/acts of the processor-implemented modules may be deployed across a number of machines, where the machines may be located in a single geographical location or distributed across a number of geographical locations.

The computer-readable program instructions can also be stored in a computer-readable storage medium that can direct one or more computer devices, programmable data processing apparatuses, and/or other devices to carry out the function/acts of the processor-implemented modules. The computer-readable storage medium containing all or partial processor-implemented modules stored therein, comprises an article of manufacture including instructions which implement aspects, operations, or steps to be performed of the function/act specified in the flowchart and/or block diagram block or blocks.

The computer-readable program instructions described herein can be downloaded to a computer-readable storage medium within a respective computing/processing device from a computer-readable storage medium. Optionally, the computer-readable program instructions can be downloaded to an external computer device or external storage device via a network. A network adapter card or network interface in each computing/processing device can receive computer-readable program instructions from the network and forward the computer-readable program instructions for permanent or temporary storage in a computer-readable storage medium within the respective computing/processing device.

The computer-readable program instructions described herein can be assembler instructions, instruction-set-architecture (ISA) instructions, machine instructions, machine dependent instructions, microcode, firmware instructions, state-setting data, or either source code or object code. The computer-readable program instructions can be written in any programming language, such as compiled or interpreted languages. In addition, the programming language can be an object-oriented programming language (e.g., “C++”), a conventional procedural programming language (e.g., “C”), or any combination thereof. The computer-readable program instructions can be distributed in any form, for example, as a stand-alone program, module, subroutine, or other unit suitable for use in a computing environment. The computer-readable program instructions can execute entirely on one computer or on multiple computers at one site or across multiple sites connected by a communication network, for example, on user's computer, partly on the user's computer, as a stand-alone software package, partly on the user's computer and partly on a remote computer, or entirely on a remote computer or server. If the computer-readable program instructions are executed entirely remotely, then the remote computer can be connected to the user's computer through any type of network, or the connection can be made to an external computer. In examples embodiments, electronic circuitry including, but not limited to, programmable logic circuitry, field-programmable gate arrays (FPGA), or programmable logic arrays (PLA) can execute the computer-readable program instructions. Electronic circuitry can utilize state information of the computer-readable program instructions to personalize the electronic circuitry in order to execute functions/acts of one or more embodiments herein.

Example embodiments described herein include logic or a number of components, modules, or mechanisms. Modules may comprise either software modules or hardware-implemented modules. A software module may be code embodied on a non-transitory machine-readable medium or in a transmission signal. A hardware-implemented module is a tangible unit capable of performing certain operations and may be configured or arranged in a certain manner. In example embodiments, one or more computer systems (e.g., a standalone computer system, a client computer system, or a server computer system) or one or more processors may be configured by software (e.g., an application or application portion) as a hardware-implemented module that operates to perform certain operations as described herein.

In example embodiments, a hardware-implemented module may be implemented mechanically or electronically. In example embodiments, hardware-implemented modules may comprise permanently configured dedicated circuitry or logic to execute certain functions/acts such as a special-purpose processor or logic circuitry (e.g., a field programmable gate array (FPGA) or an application-specific integrated circuit (ASIC)). In example embodiments, hardware-implemented modules may comprise temporary programmable logic or circuitry to perform certain functions/acts, for example, on a general-purpose processor or other programmable processor.

The term “hardware-implemented module” encompasses a tangible entity. A tangible entity may be physically constructed, permanently configured, or temporarily or transitorily configured to operate in a certain manner and/or to perform certain functions/acts described herein. Hardware-implemented modules that are temporarily configured need not be configured or instantiated at any one time. For example, if the hardware-implemented modules comprise a general-purpose processor configured using software, then the general-purpose processor may be configured as different hardware-implemented modules at different times.

Hardware-implemented modules can provide, receive, and/or exchange information from/with other hardware-implemented modules. The hardware-implemented modules herein may be communicatively coupled. Multiple hardware-implemented modules operating concurrently may communicate through signal transmission, for instance, through appropriate circuits and buses that connect the hardware-implemented modules. Multiple hardware-implemented modules configured or instantiated at different times may communicate through temporarily or permanently archived information, for instance, using the storage and retrieval of information in memory structures to which the multiple hardware-implemented modules have access. For example, one hardware-implemented module may perform an operation and store the output of that operation in a memory device to which it is communicatively coupled. Consequently, another hardware-implemented module may, at some time later, access the memory device to retrieve and process the stored information. Hardware-implemented modules may also initiate communications with input or output devices and can operate on information from the input or output devices.

In block 240, the diagnostic output comprising \dot{V}O_{2 PEAK} is transmitted (e.g., transferred) back to the user via the network 105. In example embodiments, the diagnostic output comprising \dot{V}O_{2 PEAK} is transmitted from a second computing device to a device associated with a user, and this device may be the first computing device or a third computing device. In example embodiments, the diagnostic output comprising \dot{V}O_{2 PEAK} is transmitted across a single computing device. In example embodiments, the resulting user information is stored on the data storage unit 137. In example embodiments, the resulting user information is immediately transmitted to the user's device. In example embodiments, the user's device is the single computing device. In example embodiments, the resulting user information is transmitted across the network 105 to the data acquisition system 120 for subsequent access by the user computing device 110 or the diagnostic network 130.

### Machine Learning

Machine learning is a field of study within artificial intelligence that allows computers to learn functional relationships between inputs and outputs without being explicitly programmed. Machine learning involves a module comprising algorithms that may learn from existing data by analyzing, categorizing, or identifying the data. Such machine learning algorithms operate by first constructing a model from training data to make predictions or decisions expressed as outputs. In example embodiments, the training data includes data for one or more identified features and one or more outcomes, for example, waveform data. Although example embodiments are presented with respect to a few machine learning algorithms, the principles presented herein may be applied to other machine learning algorithms.

Data supplied to a machine learning algorithm can be considered a feature, which can be described as an individual measurable property of a phenomenon being observed. The concept of a feature is related to that of an independent variable used in statistical techniques such as those used in linear regression. The performance of a machine learning algorithm in pattern recognition, classification, and regression is highly dependent on choosing informative, discriminating, and independent features. Features may comprise numerical data, categorical data, time-series data, strings, graphs, or images. Features of the disclosure may further comprise waveform data. These waveform data may include a PR interval, QRS voltages, a QT interval, and QTc-intervals, wherein the characteristic features from the QRS voltage may comprise the Q, R, or S signal, or any combination thereof. In one example embodiment, differentiating \dot{V}O_{2 PEAK} comprises distinguishing one or more characteristic features.

In general, there are two categories of machine learning problems: classification problems and regression problems. Classification problems, also referred to as categorization problems, aim at classifying items into discrete category values. Training data teach the classifying algorithm how to classify. In example embodiments, features to be categorized may include age, sex, heart rate, BMI, ventricular rate, PR intervals, QRS voltages, QT intervals, and QTc-intervals, wherein the characteristic features from the QRS voltage may comprise the Q, R, or S signal, or any combination thereof, which can be provided to the classifying machine learning algorithm and then placed into categories of, for example, the \dot{V}O_{2 PEAK}. Regression algorithms aim at quantifying and correlating one or more features. Training data teach the regression algorithm how to correlate the one or more features into a quantifiable value. In example embodiments, features such as age, sex, heart rate, BMI, ventricular rate, PR intervals, QRS voltages, QT intervals, and QTc-intervals, wherein the characteristic features from the QRS voltage may comprise the Q, R, or S signal, or any combination thereof, can be provided to the regression machine learning algorithm, resulting in one or more continuous values, for example, the \dot{V}O_{2 PEAK}.

### Embedding

In one example, the machine learning module may use embedding to provide a lower dimensional representation, such as a vector, of features to organize them based on respective similarities. In some situations, these vectors can become massive. In the case of massive vectors, particular values may become very sparse among a large number of values (e.g., a single instance of a value among 50,000 values). Because such vectors are difficult to work with, reducing the size of the vectors, in some instances, is necessary. A machine learning module can learn the embeddings along with the model parameters. In example embodiments, features such as age, sex, heart rate, BMI, ventricular rate, PR intervals, QRS voltages, QT intervals, and QTc-intervals, wherein the characteristic features from the QRS voltage may comprise the Q, R, or S signal or any combination thereof, can be mapped to vectors implemented in embedding methods. In example embodiments, embedded semantic meanings are utilized. Embedded semantic meanings are values of respective similarity. For example, the distance between two vectors, in vector space, may imply two values located elsewhere with the same distance are categorically similar. Embedded semantic meanings can be used with similarity analysis to rapidly return similar values. In example embodiments, age, sex, heart rate, PR intervals, QRS voltages, QT intervals, and QTc-intervals, wherein the characteristic features from the QRS voltage may comprise the Q, R, or S signal or any combination thereof, is embedded. In example embodiments, waveform data are embedded along with corresponding phenotypical data from one or more subject. Projections along the vector components of the ECG waveform data and the corresponding phenotypical data can be used in diagnosing \dot{V}O_{2 PEAK}. In example embodiments, the methods herein are developed to identify meaningful portions of the vector and extract semantic meanings between that space.

### Training Methods

In example embodiments, the machine learning module can be trained using techniques such as unsupervised learning, supervised learning, semi-supervised learning, reinforcement learning, transfer learning, incremental learning, curriculum learning techniques, and/or learning to learn techniques. Training typically occurs after selection and development of a machine learning module and before the machine learning module is operably in use. In one aspect, the training data used to teach the machine learning module can comprise input data, such as ECG waveform data, and the respective target output data, such as \dot{V}O_{2 PEAK}.

In an example embodiment, unsupervised learning is implemented. Unsupervised learning can involve providing all or a portion of unlabeled training data to a machine learning module. The machine learning module can then determine one or more outputs implicitly based on the provided unlabeled training data. In an example embodiment, supervised learning is implemented. Supervised learning can involve providing all or a portion of labeled training data to a machine learning module, with the machine learning module determining one or more outputs based on the provided labeled training data, and the outputs are either accepted or corrected depending on the agreement to the actual outcome of the training data. In some examples, supervised learning of machine learning system(s) can be governed by a set of rules and/or a set of labels for the training input, and the set of rules and/or set of labels may be used to correct inferences of a machine learning module.

In one example embodiment, semi-supervised learning is implemented. Semi-supervised learning can involve providing all or a portion of training data that is partially labeled to a machine learning module. During semi-supervised learning, supervised learning is used for a first portion of labeled training data, and unsupervised learning is used for a second portion of unlabeled training data. In one example embodiment, reinforcement learning is implemented. Reinforcement learning can involve first providing all or a portion of the training data to a machine learning module, and, as the machine learning module produces an output, the machine learning module receives a “reward” signal in response to a correct output. Typically, the reward signal is a numerical value, and the machine learning module is developed to maximize the numerical value of the reward signal. In addition, reinforcement learning can adopt a value function that provides a numerical value representing an expected total of the numerical values provided by the reward signal over time.

In one example embodiment, transfer learning is implemented. Transfer learning techniques can involve providing all or a portion of a first training data to a machine learning module, then, after training on the first training data, providing all or a portion of a second training data. In example embodiments, a first machine learning module can be pre-trained on data from one or more computing devices. The first trained machine learning module is then provided to a computing device, where the computing device is intended to execute the first trained machine learning model to produce an output. Then, during the second training phase, the first trained machine learning model can be additionally trained using additional training data, where the training data can be derived from kernel and non-kernel data of one or more computing devices. This second training of the machine learning module and/or the first trained machine-learning model using the training data can be performed using either supervised, unsupervised, or semi-supervised learning. In addition, it is understood transfer learning techniques can involve one, two, three, or more training attempts. Once the machine learning module has been trained on at least the training data, the training phase can be completed. The resulting trained machine learning model can be utilized as at least one of trained machine learning modules.

In one example embodiment, incremental learning is implemented. Incremental learning techniques can involve providing a trained machine learning module with input data that are used to continuously extend the knowledge of the trained machine learning module. Another machine learning training technique is curriculum learning, which can involve training the machine learning module with training data arranged in a particular order, such as providing relatively easy training examples first, then proceeding with progressively more difficult training examples. As the name suggests, the difficulty of the training data is analogous to a curriculum or course of study at a school.

In one example embodiment, learning to learn is implemented. Learning to learn, or meta-learning, comprises, in general, two levels of learning: quick learning of a single task and slower learning across many tasks. For example, a machine learning module is first trained and comprises a first set of parameters or weights. During or after operation of the first trained machine learning module, the parameters or weights are adjusted by the machine learning module. This process occurs iteratively on the success of the machine learning module. In another example, an optimizer, or another machine-learning module, is used wherein the output of a first trained machine-learning module is fed to an optimizer that constantly learns and returns the final results. Other techniques for training the machine learning module and/or the trained machine learning module are possible as well.

In an example embodiment, contrastive learning is implemented. Contrastive learning is a self-supervised model of learning in which training data are unlabeled and is considered as a form of learning in-between supervised and unsupervised learning. This method learns by contrastive loss, which separates unrelated (i.e., negative) data pairs and connects related (i.e., positive) data pairs. For example, to create positive and negative data pairs, more than one view of a datapoint, such as rotating an image or using a different time-point of a video, is used as input. Positive and negative pairs are learned by solving a dictionary look-up problem. The two views are separated into a query and a key of a dictionary. A query has a positive match to one key and a negative match to all other keys. The machine learning module then learns by connecting queries to their keys and separating queries from their non-keys. A loss function, such as those described herein, is used to minimize the distance between positive data pairs (e.g., a query to its key) while maximizing the distance between negative data points.

In some examples, after the training phase has been completed but before producing predictions expressed as outputs, a trained machine learning module can be provided to a computing device where a trained machine learning module is not already resident. In other words, after a training phase has been completed, the trained machine learning module can be downloaded to a computing device. For example, a first computing device storing a trained machine learning module can provide the trained machine learning module to a second computing device. Providing a trained machine learning module to the second computing device may comprise one or more of communicating a copy of trained machine learning module to the second computing device, making a copy of trained machine learning module for the second computing device, providing access to trained machine learning module to the second computing device, and/or otherwise providing the trained machine learning module to the second computing device. In example embodiments, a trained machine learning module can be used by the second computing device immediately after being provided by the first computing device. In some examples, after a trained machine learning module is provided to the second computing device, the trained machine learning module can be installed and/or otherwise prepared for use before the trained machine learning module can be used by the second computing device.

After a machine learning model has been trained, it can be used to output, estimate, infer, predict, generate, and/or determine. For simplicity, these terms will collectively be referred to as results. A trained machine learning module can receive input data and operably generate results. As such, the input data can be used as an input to the trained machine learning module for providing corresponding results to kernel components and non-kernel components. For example, a trained machine learning module can generate results in response to requests. In example embodiments, a trained machine learning module can be executed by a portion of other software. For example, a trained machine learning module can be executed by a result daemon to be readily available to provide results upon request.

In example embodiments, a machine learning module and/or trained machine learning module can be executed and/or accelerated using one or more computer processors and/or on-device co-processors. Such on-device co-processors can speed up training of a machine learning module and/or generation of results. In some examples, a trained machine learning module can be trained, reside, and execute to provide results on a particular computing device, and/or otherwise can make results for the particular computing device.

Input data can include data from a computing device executing a trained machine learning module and/or input data from one or more computing devices. In example embodiments, a trained machine learning module can use results as input feedback. A trained machine learning module can also rely on past results as inputs for generating new results. In example embodiments, input data can comprise waveform data and, when provided to a trained machine learning module, results in output data such as \dot{V}O_{2 PEAK}.

### Algorithms

Different machine learning algorithms have been contemplated to carry out the embodiments discussed herein. For example, neural networks (NN) (also known as artificial neural networks), convolutional neural networks, convolutional autoencoders, and deep learning NNs are contemplated. Additional algorithms have also been contemplated, such as linear regression (LiR), logistic regression (LoR), Bayesian networks (for example, naive-bayes), random forest (RF) (including decision trees), matrix factorization, a hidden Markov model (HMM), support vector machines (SVM), K-means clustering (KMC), K-nearest neighbor (KNN), a suitable statistical machine learning algorithm, and/or a heuristic machine learning system for classifying or evaluating \dot{V}O_{2 PEAK}.

### Neural Networks

In one example embodiment, Neural Networks are implemented. NNs are a family of statistical learning models influenced by biological neural networks of the brain. NNs can be trained on a relatively large dataset (e.g., 50,000 or more) and used to estimate, approximate, or predict an output that depends on a large number of inputs/features. NNs can be envisioned as so-called “neuromorphic” systems of interconnected processor elements, or “neurons,” and exchange electronic signals, or “messages.” Similar to the so-called “plasticity” of synaptic neurotransmitter connections that carry messages between biological neurons, the connections in NNs that carry electronic “messages” between “neurons” are provided with numeric weights that correspond to the strength or weakness of a given connection. The weights can be tuned based on experience, making NNs adaptive to inputs and capable of learning. For example, an NN for determining \dot{V}O_{2 PEAK} is defined by a set of input neurons that can be given input data such as ECG waveform data. The input neuron weighs and transforms the input data and passes the result to other neurons, often referred to as “hidden” neurons. This is repeated until an output neuron is activated. The activated output neuron produces a result. In example embodiments, ECG waveform data are used to train the neurons in a NN machine learning module, which, after training, is used to estimate \dot{V}O_{2 PEAK}.

### Convolutional Neural Network (CNN)

In an example embodiment, a convolutional neural network is implemented. CNNs are a class of NNs further attempting to replicate the biological neural networks, but of the animal visual cortex. CNNs process data with a grid pattern to learn spatial hierarchies of features. Wherein NNs are highly connected, sometimes fully connected, CNNs are connected such that neurons corresponding to neighboring pixels are connected. This significantly reduces the number of weights and calculations each neuron must perform.

In general, input data, such as ECG waveform data, comprise a multidimensional vector. A CNN typically comprises three layers: convolution, pooling, and fully connected. The convolution and pooling layers extract features, and the fully connected layer combines the extracted features into an output.

In particular, the convolutional layer comprises multiple mathematical operations such as linear operations, a specialized type being a convolution. The convolutional layer calculates the scalar product between the weights and the region connected to the input volume of the neurons. These computations are performed on kernels, which are reduced dimensions of the input vector. The kernels span the entirety of the input. The rectified linear unit (i.e., ReLU) applies an elementwise activation function (e.g., sigmoid function) on the kernels.

CNNs can be optimized with hyperparameters. In general, three hyperparameters are used: depth, stride, and zero-padding. Depth controls the number of neurons within a layer. Reducing the depth may increase the speed of the CNN but may also reduce the accuracy of the CNN. Stride determines the overlap of the neurons. Zero-padding controls the border padding in the input.

The pooling layer down-samples along the spatial dimensionality of the given input (i.e., convolutional layer output), reducing the number of parameters within that activation. As an example, kernels are reduced to dimensionalities of 2×2 with a stride of 2, which scales the activation map down to 25%. The fully connected layer uses inter-layer-connected neurons (i.e., neurons are only connected to neurons in other layers) to score the activations for classification and/or regression. Extracted features may become hierarchically more complex as one layer feeds its output into the next layer.

In example embodiments, the CNN is pre-trained. A pre-trained machine learning model is a model that has been previously trained to solve a similar problem. The pre-trained machine learning model is generally pre-trained with similar input data to that of the new problem. A pre-trained machine learning model further trained to solve a new problem is generally referred to as transfer learning, which is described herein. In some instances, a pre-trained machine learning model is trained on a large dataset of related information. The pre-trained model is then further trained and tuned for the new problem. Using a pre-trained machine learning module provides the advantage of building a new machine learning module with input neurons/nodes that are already familiar with the input data and are more readily refined to a particular problem. For example, a CNN previously trained using ECG waveform data may be further trained to estimate \dot{V}O_{2 PEAK}. While the pre-trained CNN of Diamant was used to train for variables already recognized as derivable from ECG data (e.g., sex, BMI, etc.), the pre-trained CNN was further developed herein to estimate \dot{V}O_{2 PEAK}. To the best of Applicants' knowledge, no other machine learning model, including the pre-trained CNN of Diamant, has been capable of estimating \dot{V}O_{2 PEAK} based on ECG data.

### Convolutional Autoencoder

In example embodiments, convolutional autoencoder (CAE) is implemented. A CAE is a type of neural network and comprises, in general, two main components: a convolutional operator that filters an input signal to extract features of the signal, and an autoencoder that learns a set of signals from an input and reconstructs the signal into an output. By combining these two components, the CAE learns the optimal filters that minimize reconstruction error resulting an improved output. CAEs are trained to only learn filters capable of feature extraction that can be used to reconstruct the input. Generally, convolutional autoencoders implement unsupervised learning. In example embodiments, the convolutional autoencoder is a variational convolutional autoencoder. In example embodiments, features from an ECG waveform data are used as an input signal into a CAE, which reconstructs that signal into an output such as \dot{V}O_{2 PEAK}.

### Deep Learning

In example embodiments, deep learning is implemented. Deep learning expands the neural network by including more layers of neurons. A deep learning module is characterized as having three “macro” layers: (1) an input layer that takes in the input features and fetches embeddings for the input, (2) one or more intermediate (or hidden) layers that introduces nonlinear neural net transformations to the inputs, and (3) a response layer that transforms the final results of the intermediate layers to the prediction. In example embodiments, ECG waveform data are used to train the neurons of a deep learning module, which, after training, is used to estimate \dot{V}O_{2 PEAK}.

### Recurrent Neural Network (RNN)

In an example embodiment, a recurrent neural network is implemented. RNNs are a class of NNs further attempting to replicate the biological neural networks of the brain. RNNs comprise delay differential equations on sequential data or time series data to replicate the processes and interactions of the human brain. RNNs have “memory,” wherein the RNN can take information from prior inputs to influence the current output. RNNs can process variable length sequences of inputs by using their “memory” or internal state information. Where NNs may assume inputs are independent from the outputs, the outputs of RNNs may be dependent on prior elements with the input sequence.

### Long Short-Term Memory (LSTM)

In an example embodiment, a Long Short-term Memory is implemented. LSTM are a class of RNNs designed to overcome vanishing and exploding gradients. In RNNs, long term dependencies become more difficult to capture because the parameters or weights either do not change with training or fluctuate rapidly. This occurs when the RNN gradient exponentially decreases to zero, resulting in no change to the weights or parameters, or exponentially increases to infinity, resulting in large changes in the weights or parameters. This exponential effect is dependent on the number of layers and multiplicative gradient. LSTM overcomes the vanishing/exploding gradients by implementing “cells” within the hidden layers of the NN. The “cells” comprise three gates: an input gate, an output gate, and a forget gate. The input gate reduces error by controlling relevant inputs to update the current cell state. The output gate reduces error by controlling relevant memory content in the present hidden state. The forget gate reduces error by controlling whether prior cell states are put in “memory” or forgotten. The gates use activation functions to determine whether the data can pass through the gates. While one skilled in the art would recognize the use of any relevant activation function, example activation functions are sigmoid, tanh, and ReLU.

### Linear Regession (LiR)

In one example embodiment, linear regression machine learning is implemented. LiR is typically used in machine learning to predict a result through the mathematical relationship between an independent and dependent variable, such as ECG waveform data and \dot{V}O_{2 PEAK}, respectively. A simple linear regression model would have one independent variable (x) and one dependent variable (y). A representation of an example mathematical relationship of a simple linear regression model would be y=mx+b. In this example, the machine learning algorithm tries variations of the tuning variables m and b to optimize a line that includes all the given training data.

The tuning variables can be optimized, for example, with a cost function. A cost function takes advantage of the minimization problem to identify the optimal tuning variables. The minimization problem proposes the optimal tuning variable will minimize the error between the predicted outcome and the actual outcome. An example cost function may comprise summing all the square differences between the predicted and actual output values and dividing them by the total number of input values and results in the average square error.

To select new tuning variables to reduce the cost function, the machine learning module may use, for example, gradient descent methods. An example gradient descent method comprises evaluating the partial derivative of the cost function with respect to the tuning variables. The sign and magnitude of the partial derivatives indicate whether the choice of a new tuning variable value will reduce the cost function, thereby optimizing the linear regression algorithm. A new tuning variable value is selected depending on a set threshold. Depending on the machine learning module, a steep or gradual negative slope is selected. Both the cost function and gradient descent can be used with other algorithms and modules mentioned throughout. For the sake of brevity, both the cost function and gradient descent are well known in the art and are applicable to other machine learning algorithms and may not be mentioned with the same detail.

LiR models may have many levels of complexity comprising one or more independent variables. Furthermore, in an LiR function with more than one independent variable, each independent variable may have the same one or more tuning variables or each, separately, may have their own one or more tuning variables. The number of independent variables and tuning variables will be understood by one skilled in the art for the problem being solved. In example embodiments, ECG waveform data are used as the independent variables to train a LiR machine learning module, which, after training, is used to estimate, for example, \dot{V}O_{2 PEAK}.

### Logistic Regression (LoR)

In one example embodiment, logistic regression machine learning is implemented. Logistic Regression, often considered a LiR type model, is typically used in machine learning to classify information, such as ECG waveform data into categories such as \dot{V}O_{2 PEAK}. LoR takes advantage of probability to predict an outcome from input data. However, what makes LoR different from a LiR is that LoR uses a more complex logistic function, for example a sigmoid function. In addition, the cost function can be a sigmoid function limited to a result between 0 and 1. For example, the sigmoid function can be of the form f(x)=1/(1+e^{-x}), where x represents some linear representation of input features and tuning variables. Similar to LiR, the tuning variable(s) of the cost function are optimized (typically by taking the log of some variation of the cost function) such that the result of the cost function, given variable representations of the input features, is a number between 0 and 1, preferably falling on either side of 0.5. As described in LiR, gradient descent may also be used in LoR cost function optimization and is an example of the process. In example embodiments, ECG waveform data are used as the independent variables to train a LoR machine learning module, which, after training, is used to estimate, for example, \dot{V}O_{2 PEAK}.

### Bayesian Network (BN)

In one example embodiment, a Bayesian Network is implemented. BNs are used in machine learning to make predictions through Bayesian inference from probabilistic graphical models. In BNs, input features are mapped onto a directed acyclic graph forming the nodes of the graph. The edges connecting the nodes contain the conditional dependencies between nodes to form a predicative model. For each connected node, the probability of the input features resulting in the connected node is learned and forms the predictive mechanism. The nodes may comprise the same, similar, or different probability functions to determine movement from one node to another. The nodes of a Bayesian network are conditionally independent of its non-descendants given its parents thus satisfying a local Markov property. This property affords reduced computations in larger networks by simplifying the joint distribution.

There are multiple methods to evaluate the inference, or predictability, in a BN, but only two are mentioned for demonstrative purposes. The first method involves computing the joint probability of a particular assignment of values for each variable. The joint probability can be considered the product of each conditional probability and, in some instances, comprises the logarithm of that product. The second method is Markov chain Monte Carlo (MCMC), which can be implemented when the sample size is large. MCMC is a well-known class of sample distribution algorithms and will not be discussed in detail herein.

The assumption of conditional independence of variables forms the basis for Naïve Bayes classifiers. This assumption implies there is no correlation between different input features. As a result, the number of computed probabilities is significantly reduced as well as the computation of the probability normalization. While independence between features is rarely true, this assumption exchanges reduced computations for less accurate predictions, however the predictions are reasonably accurate. In example embodiments, ECG waveform data are mapped to the BN graph to train the BN machine learning module, which, after training, is used to estimate \dot{V}O_{2 PEAK}.

### Random Forest

In one example embodiment, random forest is implemented. RF consists of an ensemble of decision trees producing individual class predictions. The prevailing prediction from the ensemble of decision trees becomes the RF prediction. Decision trees are branching flowchart-like graphs comprising of the root, nodes, edges/branches, and leaves. The root is the first decision node from which feature information is assessed and from it extends the first set of edges/branches. The edges/branches contain the information of the outcome of a node and pass the information to the next node. The leaf nodes are the terminal nodes that output the prediction. Decision trees can be used for both classification as well as regression and is typically trained using supervised learning methods. Training of a decision tree is sensitive to the training data set. An individual decision tree may become over or under-fit to the training data and result in a poor predictive model. Random forest compensates by using multiple decision trees trained on different data sets. In example embodiments, ECG waveform data are used to train the nodes of the decision trees of a RF machine learning module, which, after training, is used to estimate \dot{V}O_{2 PEAK}.

### Gradient Boosting

In an example embodiment, gradient boosting is implemented. Gradient boosting is a method of strengthening the evaluation capability of a decision tree node. In general, a tree is fit on a modified version of an original data set. For example, a decision tree is first trained with equal weights across its nodes. The decision tree is allowed to evaluate data to identify nodes that are less accurate. Another tree is added to the model and the weights of the corresponding underperforming nodes are then modified in the new tree to improve their accuracy. This process is performed iteratively until the accuracy of the model has reached a defined threshold or a defined limit of trees has been reached. Less accurate nodes are identified by the gradient of a loss function. Loss functions must be differentiable such as a linear or logarithmic functions. The modified node weights in the new tree are selected to minimize the gradient of the loss function. In an example embodiment, a decision tree is implemented to determine \dot{V}O_{2 PEAK}, and gradient boosting is applied to the tree to improve its ability to accurately determine the \dot{V}O_{2 PEAK}.

### Matrix Factorization

In example embodiments, Matrix Factorization is implemented. Matrix factorization machine learning exploits inherent relationships between two entities drawn out when multiplied together. Generally, the input features are mapped to a matrix F, which is multiplied with a matrix R containing the relationship between the features and a predicted outcome. The resulting dot product provides the prediction. The matrix R is constructed by assigning random values throughout the matrix. In this example, two training matrices are assembled. The first matrix X contains training input features, and the second matrix Z contains the known output of the training input features. First, the dot product of R and X are computed, and the mean squared error, as one example method, of the result is estimated. The values in R are modulated, and the process is repeated in a gradient descent-style approach until the error is appropriately minimized. The trained matrix R is then used in the machine learning model. In example embodiments, ECG waveform data are used to train the relationship matrix R in a matrix factorization machine learning module. After training, the relationship matrix R and input matrix F, which comprises vector representations of the ECG waveform data, results in the prediction matrix P comprising \dot{V}O_{2 PEAK}.

### Hidden Markov Model (HMM)

In example embodiments, a hidden Markov model is implemented. An HMM takes advantage of the statistical Markov model to predict an outcome. A Markov model assumes a Markov process, wherein the probability of an outcome is solely dependent on the previous event. In the case of HMM, it is assumed an unknown or “hidden” state is dependent on some observable event. An HMM comprises a network of connected nodes. Traversing the network is dependent on three model parameters: start probability, state transition probabilities, and observation probability. The start probability is a variable that governs, from the input node, the most plausible consecutive state. From there, each node i has a state transition probability to node j. Typically, the state transition probabilities are stored in a matrix Mᵢⱼ, wherein the sum of the rows, representing the probability of state i transitioning to state j, equals 1. The observation probability is a variable containing the probability of output o occurring. These, too, are typically stored in a matrix Nₒⱼ, wherein the probability of output o is dependent on state j. To build the model parameters and train the HMM, the state and output probabilities are computed. This can be accomplished with, for example, an inductive algorithm. Next, the state sequences are ranked on probability, which can be accomplished, for example, with the Viterbi algorithm. Finally, the model parameters are modulated to maximize the probability of a certain sequence of observations. This is typically accomplished with an iterative process wherein the neighborhood of states is explored, the probabilities of the state sequences are measured, and model parameters are updated to increase the probabilities of the state sequences. In example embodiments, ECG waveform data are used to train the nodes/states of the HMM machine learning module, which, after training, is used to estimate \dot{V}O_{2 PEAK}.

### Support Vector Machine (SVM)

In example embodiments, support vector machines are implemented. SVMs separate data into classes defined by n-dimensional hyperplanes (n-hyperplane) and are used in both regression and classification problems. Hyperplanes are decision boundaries developed during the training process of an SVM. The dimensionality of a hyperplane depends on the number of input features. For example, an SVM with two input features will have a linear (1-dimensional) hyperplane, while an SVM with three input features will have a planer (2-dimensional) hyperplane. A hyperplane is optimized to have the largest margin or spatial distance from the nearest data point for each data type. In the case of simple linear regression and classification, a linear equation is used to develop the hyperplane. However, when the features are more complex, a kernel is used to describe the hyperplane. A kernel is a function that transforms the input features into a higher dimensional space. Kernel functions can be linear, polynomial, a radial distribution function (or gaussian radial distribution function), or sigmoidal. In example embodiments, ECG waveform data are used to train the linear equation or kernel function of the SVM machine learning module, which, after training, is used to estimate \dot{V}O_{2 PEAK}.

### K-Means Clustering (KMC)

In one example embodiment, K-means clustering is implemented. KMC assumes data points have implicit shared characteristics and “clusters” data within a centroid or “mean” of the clustered data points. During training, KMC adds a number of k centroids and optimizes its position around clusters. This process is iterative, where each centroid, initially positioned at random, is re-positioned towards the average point of a cluster. This process concludes when the centroids have reached an optimal position within a cluster. Training of a KMC module is typically unsupervised. In example embodiments, ECG waveform data are used to train the centroids of a KMC machine learning module, which, after training, is used to estimate \dot{V}O_{2 PEAK}.

### K-Nearest Neighbor (KNN)

In one example embodiment, K-nearest neighbor is implemented. On a general level, KNN shares similar characteristics to KMC. For example, KNN assumes data points near each other share similar characteristics and computes the distance between data points to identify those similar characteristics, but instead of k centroids, KNN uses k number of neighbors. The k in KNN represents how many neighbors will assign a data point to a class (for classification) or object property value (for regression). Selection of an appropriate number for k is integral to the accuracy of KNN. For example, a large k may reduce random error associated with variance in the data but increase error by ignoring small but significant differences in the data. Therefore, a careful choice of k is selected to balance overfitting and underfitting. Concluding whether some data point belongs to some class or property value k, the distance between neighbors is computed. Common methods to compute this distance are Euclidean, Manhattan, or Hamming, to name a few. In some embodiments, neighbors are given weights depending on the neighbor distance to scale the similarity between neighbors to reduce the error of edge neighbors of one class “out-voting” near neighbors of another class. In one example embodiment, k is 1, and a Markov model approach is utilized. In example embodiments, ECG waveform data are used to train a KNN machine learning module, which, after training, is used to estimate \dot{V}O_{2 PEAK}.

To perform one or more of its functionalities, the machine learning module may communicate with one or more other systems. For example, an integration system may integrate the machine learning module with one or more email servers, web servers, one or more databases, or other servers, systems, or repositories. In addition, one or more functionalities may require communication between a user and the machine learning module.

Any one or more of the modules described herein may be implemented using hardware (e.g., one or more processors of a computer/machine) or a combination of hardware and software. For example, any module described herein may configure a hardware processor (e.g., among one or more hardware processors of a machine) to perform the operations described herein for that module. In some example embodiments, any one or more of the modules described herein may comprise one or more hardware processors and may be configured to perform the operations described herein. In certain example embodiments, one or more hardware processors are configured to include any one or more of the modules described herein.

Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices. The multiple machines, databases, or devices are communicatively coupled to enable communications between the multiple machines, databases, or devices. The modules themselves are communicatively coupled (e.g., via appropriate interfaces) to each other and to various data sources to allow information to be passed between the applications and allow the applications to share and access common data.

### Example Computing Device

FIG. 16 depicts a block diagram of a computing machine 2000 and a module 2050 in accordance with certain examples. The computing machine 2000 may comprise, but is not limited to, remote devices, work stations, servers, computers, general purpose computers, Internet/web appliances, hand-held devices, wireless devices, portable devices, wearable computers, cellular or mobile phones, personal digital assistants (PDAs), smart phones, smart watches, tablets, ultrabooks, netbooks, laptops, desktops, multi-processor systems, microprocessor-based or programmable consumer electronics, game consoles, set-top boxes, network PCs, mini-computers, and any machine capable of executing the instructions. The module 2050 may comprise one or more hardware or software elements configured to facilitate the computing machine 2000 in performing the various methods and processing functions presented herein. The computing machine 2000 may include various internal or attached components such as a processor 2010, a system bus 2020, a system memory 2030, storage media 2040, an input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The one or more processor 2010 may be configured to execute code or instructions to perform the operations and functionality described herein, manage a request flow and address mappings, and to perform calculations and generate commands. Such code or instructions could include, but is not limited to, firmware, resident software, microcode, and the like. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (ASIC), tensor processing units (TPUs), a graphics processing unit (GPU), a field programmable gate array (FPGA), a programmable logic device (PLD), a radio-frequency integrated circuit (RFIC), a controller, a state machine, gated logic, discrete hardware components, any other processing unit, or any combination or multiplicity thereof. In example embodiments, each processor 2010 can include a reduced instruction set computer (RISC) microprocessor. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines. Processors 2010 are coupled to system memory and various other components via a system bus 2020.

The system memory 2030 may include non-volatile memories such as read-only memory (ROM), programmable read-only memory (PROM), erasable programmable read-only memory (EPROM), flash memory, or any other device capable of storing program instructions or data with or without applied power. The system memory 2030 may also include volatile memories such as random-access memory (RAM), static random-access memory (SRAM), dynamic random-access memory (DRAM), and synchronous dynamic random-access memory (SDRAM). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 is coupled to system bus 2020 and can include a basic input/output system (BIOS), which controls certain basic functions of the processor 2010 and/or operate in conjunction with, a non-volatile storage device such as the storage media 2040.

In example embodiments, the computing machine 2000 includes a graphics processing unit (GPU) 2090. Graphics processing unit 2090 is a specialized electronic circuit designed to manipulate and alter memory to accelerate the creation of images in a frame buffer intended for output to a display. In general, a graphics processing unit 2090 is efficient at manipulating computer graphics and image processing and has a highly parallel structure that makes it more effective than general-purpose CPUs for algorithms where processing of large blocks of data is done in parallel.

The storage media 2040 may include a hard disk, a floppy disk, a compact disc read only memory CD-ROM), a digital versatile disc (DVD), a Blu-ray disc, a magnetic tape, a flash memory, other non-volatile memory device, a solid state drive (SSD), any magnetic storage device, any optical storage device, any electrical storage device, any electromagnetic storage device, any semiconductor storage device, any physical-based storage device, any removable and non-removable media, any other data storage device, or any combination or multiplicity thereof. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The module 2050 may comprise one or more hardware or software elements, as well as an operating system, configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer-readable media on which instructions or code may be stored for execution by the processor 2010. Machine or computer-readable media may generally refer to any medium or media used to provide instructions to the processor 2010. Such machine or computer-readable media associated with the module 2050 may comprise a computer software product. Each of the operating system, one or more application programs, other program modules, and program data or some combination thereof, may include an implementation of a networking environment. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (I/O) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for coupling in operation the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (SCSI), serial-attached SCSI (SAS), fiber channel, peripheral component interconnect (PCI), PCI express (PCIe), serial bus, parallel bus, advanced technology attached (ATA), serial ATA (SATA), universal serial bus (USB), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including cursor control devices, touchscreens, scanners, electronic digitizers, sensors, receivers, touchpads, trackballs, cameras, microphones, alphanumeric input devices, any other pointing devices, or any combinations thereof. The I/O interface 2060 may couple the computing machine 2000 to various output devices including video displays (the computing machine 2000 may further include a graphics display, for example, a plasma display panel (PDP), a light emitting diode (LED) display, a liquid crystal display (LCD), a projector, a cathode ray tube (CRT), or any other display capable of displaying graphics or video), audio generation device, printers, projectors, tactile feedback devices, automation control, robotic components, actuators, motors, fans, solenoids, valves, pumps, transmitters, signal emitters, lights, and so forth. The I/O interface 2060 may couple the computing machine 2000 to various devices capable of input and output, such as a storage unit. The devices can be interconnected to the system bus 2020 via a user interface adapter, which can include, for example, a Super I/O chip integrating multiple device adapters into a single integrated circuit.

The computing machine 2000 may operate in a networked environment using logical connections through the network interface 2070 to one or more other systems or computing machines across the network 2080. The network 2080 may include a local area network (LAN), a wide area network (WAN), an intranet, an Internet, a mobile telephone network, a storage area network (SAN), a personal area network (PAN), a metropolitan area network (MAN), a wireless network (WiFi), wireless access networks, a wireless local area network (WLAN), a virtual private network (VPN), a cellular or other mobile communication network, Bluetooth, near field communication (NFC), ultra-wideband, wired networks, telephone networks, optical networks, copper transmission cables, or combinations thereof or any other appropriate architecture or system that facilitates the communication of signals and data. The network 2080 may be packet switched, circuit switched, of any topology, and may use any communication protocol. The network 2080 may comprise routers, firewalls, switches, gateway computers and/or edge servers. Communication links within the network 2080 may involve various digital or analog communication media such as fiber optic cables, free-space optics, waveguides, electrical conductors, wireless links, antennas, radio-frequency communications, and so forth.

Information for facilitating reliable communications can be provided, for example, as packet/message sequencing information, encapsulation headers and/or footers, size/time information, and transmission verification information such as cyclic redundancy check (CRC) and/or parity check values. Communications can be made encoded/encrypted, or otherwise made secure, and/or decrypted/decoded using one or more cryptographic protocols and/or algorithms, such as, but not limited to, Data Encryption Standard (DES), Advanced Encryption Standard (AES), a Rivest-Shamir-Adelman (RSA) algorithm, a Diffie-Hellman algorithm, a secure sockets protocol such as Secure Sockets Layer (SSL) or Transport Layer Security (TLS), and/or Digital Signature Algorithm (DSA). Other cryptographic protocols and/or algorithms can be used as well or in addition to those listed herein to secure and then decrypt/decode communications.

The processor 2010 may be connected to the other elements of the computing machine 2000 or the various peripherals discussed herein through the system bus 2020. The system bus 2020 represents one or more of any of several types of bus structures, including a memory bus or memory controller, a peripheral bus, an accelerated graphics port, and a processor or local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system-on-chip (SOC), a system-on-package (“SOP”), or an ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those ordinarily skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

A “server” may comprise a physical data processing system (for example, the computing machine 2000 as shown in FIG. 16) running a server program. A physical server may or may not include a display and keyboard. A physical server may be connected, for example by a network, to other computing devices. Servers connected via a network may operate in the capacity of a server machine or a client machine in a server-client network environment, or as a peer machine in a distributed (e.g., peer-to-peer) network environment. The computing machine 2000 can include clients' servers. For example, a client and server can be remote from each other and interact through a network. The relationship of client and server arises by virtue of computer programs in communication with each other, running on the respective computers.

The example systems, methods, and acts described in the examples and described in the figures presented previously are illustrative, not intended to be exhaustive, and not meant to be limiting. In alternative examples, certain acts can be performed in a different order, in parallel with one another, omitted entirely, and/or combined between different examples, and/or certain additional acts can be performed, without departing from the scope and spirit of various examples. Plural instances may implement components, operations, or structures described as a single instance. Structures and functionality that may appear as separate in example embodiments may be implemented as a combined structure or component. Similarly, structures and functionality that may appear as a single component may be implemented as separate components. Accordingly, such alternative examples are included in the scope of the following claims, which are to be accorded the broadest interpretation to encompass such alternate examples. The terminology used herein was chosen to best explain the principles of the embodiments, the practical application, or technical improvement over technologies found in the marketplace, or to enable others of ordinary skill in the art to understand the embodiments disclosed herein.

Further embodiments are illustrated in the following Example, which is given for illustrative purposes only and is not intended to limit the scope of the disclosure.

### EXAMPLE

### Example 1—ECG Deep Learning Predicts Exercise Peak \dot{V}O₂, Cardiovascular Outcomes and Future Death

### Introduction

Oxygen consumption at peak exercise (\dot{V}O_{2 PEAK}) is an integrative marker of cardiorespiratory fitness (CRF) and is among the most powerful prognostic metrics of human health. Across diverse populations, \dot{V}O_{2 PEAK} is a robust and independent predictor of overall mortality, multiple adverse cardiovascular outcomes, and a wide range of other important conditions including malignancy, diabetes, dementia, and many others. However, accurate assessment of \dot{V}O_{2 PEAK} requires dedicated cardiopulmonary exercise testing (CPET), which is resource-intensive, dependent on interpretation expertise, and not universally available. As a result, despite its broad utility, quantified CRF is currently not routinely assessed in clinical practice, with the most common clinical use of \dot{V}O_{2 PEAK} occurring in the relatively narrow area of clinical assessment of patients with advanced heart failure to guide evaluation for advanced therapies including heart transplantation. However, its predictive value extends into disease-free populations.

Recent work suggests that deep learning models utilizing resting 12-lead electrocardiograms (ECGs) can extract rich elements of cardiac structure, function, and even future disease susceptibility. Applicants show that \dot{V}O_{2 PEAK} may be similarly estimable using resting 12-lead ECG, thereby providing automated, instantaneous, and widespread access to this important metric.

In the current study, Applicants assessed whether automated ECG feature analysis, using both assessment of standard ECG intervals and a deep learning model, can be used to estimate \dot{V}O_{2 PEAK} in approximately 2,300 individuals who underwent CPET. In a separate ambulatory dataset of over 80,000 individuals, Applicants further tested whether 12-lead ECG-derived estimates of \dot{V}O_{2 PEAK} predict future clinical outcomes.

### Methods

### Data Availability

Massachusetts General Hospital (MGH) data contain protected health information and cannot be shared publicly. ECG pre-processing code for the PCLR model is available at https://github.com/broadinstitute/m14h/tree/master/model_zoo/PCLR. Data processing scripts used to perform the analyses described herein are available at https://github.com/shaankhurshid/vo2_prediction.

### Study Samples

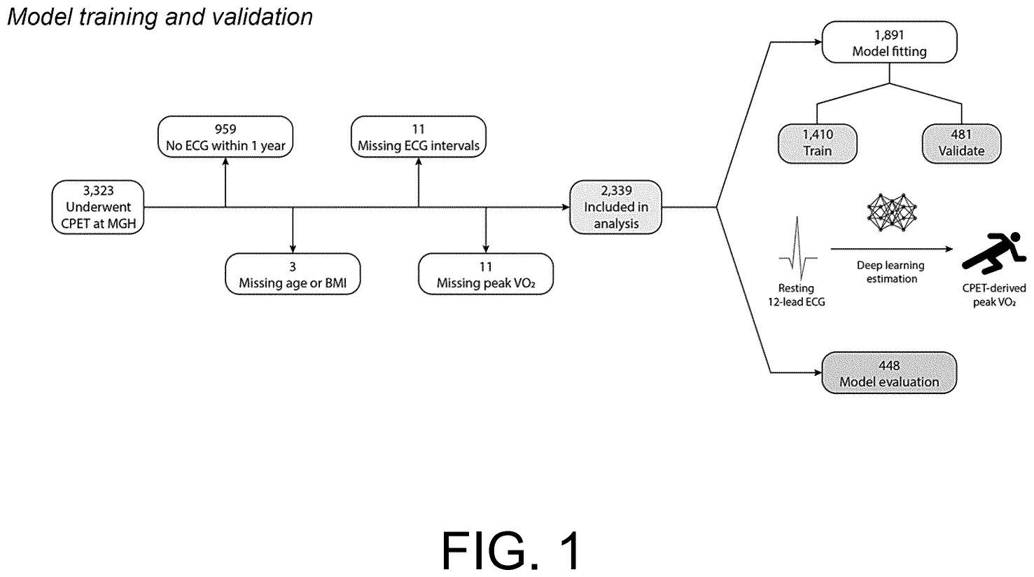

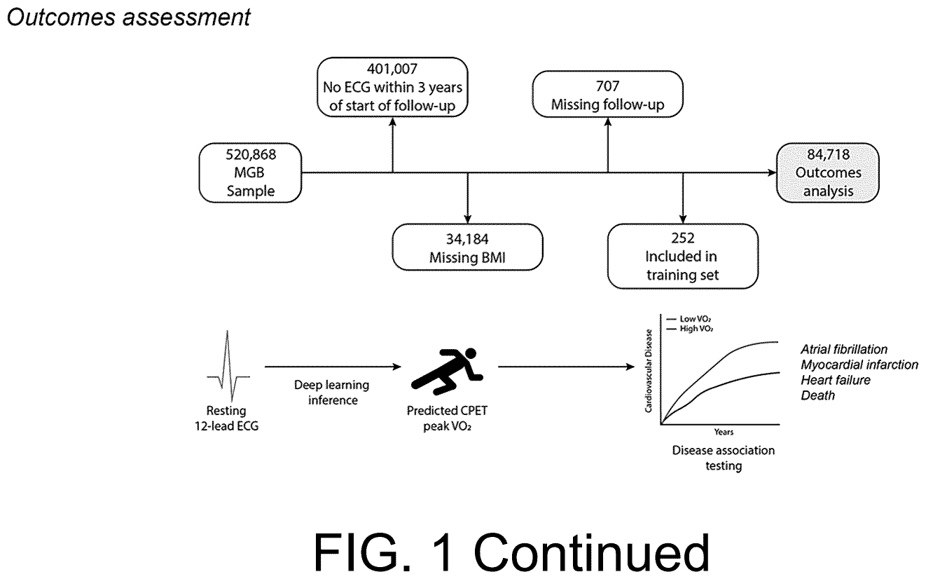

Applicants estimated 12-lead ECG-derived \dot{V}O_{2 PEAK} in a sample of 2,339 individuals who underwent clinically indicated CPET testing in the MGH outpatient CPET laboratory between Oct. 1, 2011 and Sep. 10, 2021 and had at least one 12-lead ECG performed within one year of the CPET study (FIG. 1). For individuals with multiple ECGs within one year of the CPET, Applicants selected the tracing closest to the CPET for \dot{V}O_{2 PEAK} estimation.

Applicants assessed associations between 12-lead ECG-derived \dot{V}O_{2 PEAK} estimates and incident disease in the Community Care Cohort Project (C3PO), a sample of 520,868 individuals receiving longitudinal primary care in the Mass General Brigham (MGB) healthcare system. The current analysis included 84,718 individuals who underwent a 12-lead ECG within 3 years prior to the start of cohort follow-up (FIG. 1). Use of MGB data were approved by the MGB Institutional Review Board.

### Cardiopulmonary Exercise Testing (CPET)

All patients performed a maximal effort graded exercise test with continuous measurement of gas exchange. Testing was performed on treadmill (Woodway Pro 27, Woodway USA, Waukesha, WI), upright cycle ergometer (Sport Excalibur Bicycle Ergometer, Lode, Holland), or rowing ergometer (Concept2 PM4, Concept2, Morrisville, VT), with modality jointly chosen by subject and supervising exercise physiologist. All tests proceeded until exhaustion, the onset of limiting symptoms, or the development of a clinical contraindication to continued exercise. Test protocols have been described in detail previously; briefly, workload was increased on a continuous or near-continuous basis on each exercise modality to target exercise duration of 10-12 minutes. All tests were performed with real-time 12-lead ECG monitoring (Mortara Instrument X12+ wireless ECG transmitter, Milwaukee, WI). Gas exchange was measured on a breath-by-breath basis using a Hans Rudolph V2 Mask (Hans Rudolph, Inc, Shawnee, KS), a commercially available metabolic cart and gas exchange analyzer (Ultima CardiaO2; Medgraphics Diagnostics, St. Paul, MN) and analyzed using Breeze Suite software (Medgraphics Diagnostics, Version 8.2, 2015). Peak oxygen consumption (\dot{V}O_{2 PEAK}) was defined as the highest oxygen uptake, averaged over a period of 30 seconds, during the last minute of symptom-limited exercise.

### Clinical Outcome Ascertainment

Outcomes included atrial fibrillation (AF), myocardial infarction (MI), heart failure (HF), and all-cause death. AF was defined using a previously validated electronic health record-based AF classification scheme (PPV 92%). MI was defined using the presence of ≥2 ICD-9 or ICD-10 codes using previously validated code sets (PPV≥85%). HF was defined using the presence of ≥1 ICD-9 or ICD-10 code applied in the inpatient setting using a previously published code set. Death information was obtained either from the Social Security Death Index or MGB internal documentation of death. Details of outcome definitions are provided in Table 1.

TABLE 1
Disease outcome definitions

| | Code | | |
|---|---|---|---|
| Phenotype | type | Data codes | Data code definitions |
| Atrial | ICD9 | 427.3, 427.31, | (Atrial fibrillation and flutter, atrial fibrillation, atrial flutter, atrial cardioversion |
| Fibril- | | 427.32, 99.61 | |
| lation | | | |
| Atrial | ICD10 | I48.0, I48.1, | Paroxysmal atrial fibrillation, Persistent atrial fibrillation, Chronic atrial fibrillation, |
| Fibril- | | I48.2, I48.3, | Typical atrial flutter, Atypical atrial flutter, Unspecified atrial fibrillation, Unspecified |
| lation | | I48.4, I48.91, | atrial flutter |
| | | I48.92 | |
| Atrial | CPT | 33253, 33257, | Operative incisions and reconstruction of atria for treatment of atrial fibrillation or atrial |
| Fibril- | | 33259, 92960, | flutter (e.g., maze procedure), Operative tissue ablation and reconstruction of atria, performed |
| lation | | 92961, 93650, | at the time of other cardiac procedure(s), limited (e.g., modified maze procedure), Operative |
| | | 93656, 93657 | tissue ablation and reconstruction of atria, performed at the time of other cardiac procedure(s), |
| | | | extensive (e.g., maze procedure), with cardiopulmonary bypass (List separately in addition to |
| | | | code for primary procedure), Cardioversion, elective, electrical conversion of arrhythmia; |
| | | | external, Cardioversion, elective, electrical conversion of arrhythmia; internal (separate |
| | | | procedure), Intracardiac catheter ablation of atrioventricular node function, atrioventricular |
| | | | conduction for creation of complete heart block, with or without temporary pacemaker placement, |
| | | | Comprehensive electrophysiologic evaluation, including transseptal catheterizations, insertion |
| | | | and repositioning of multiple electrode catheters with induction or attempted induction of an |
| | | | arrhythmia with atrial recording and pacing, when possible, right ventricular pacing and |
| | | | recording, His bundle recording with intracardiac catheter ablation of arrhythmogenic focus, with |
| | | | treatment of atrial fibrillation by ablation by pulmonary vein isolation, Additional linear or |
| | | | focal intracardiac catheter ablation of the left or right atrium for treatment of atrial |
| | | | fibrillation remaining after completion of pulmonary vein isolation |
| Heart | ICD9 | 398.91, 402.01, | Rheumatic heart failure (congestive), Malignant hypertensive heart disease with heart failure, |
| Failure | | 402.11, 402.91, | Benign hypertensive heart disease with heart failure, Unspecified hypertensive heart disease |
| | | 404.01, 404.03, | with heart failure, Hypertensive heart and chronic kidney disease, malignant, with heart failure |
| | | 404.11, 404.13, | and with chronic kidney disease stage I through stage IV, or unspecified, Hypertensive heart and |
| | | 404.91, 404.93, | chronic kidney disease, malignant, with heart failure and with chronic kidney disease stage V or |
| | | 428, 428.1, | end stage renal disease, Hypertensive heart and chronic kidney disease, benign, with heart |
| | | 428.2, 428.21, | failure and with chronic kidney disease stage I through stage IV, or unspecified, Hypertensive |
| | | 428.22, 428.23, | heart and chronic kidney disease, benign, with chronic kidney disease, unspecified, with heart |
| | | 428.3, 428.31, | failure and with chronic kidney disease stage I through stage IV, or unspecified, Hypertensive |
| | | 428.32, 428.33, | heart and chronic kidney disease, unspecified, with heart failure and chronic kidney disease |
| | | 428.4, 428.41, | stage V or end stage renal disease, Congestive heart failure, unspecified, Left heart failure, |
| | | 428.42, 428.43, | Systolic heart failure, unspecified, Acute systolic heart failure, Chronic systolic heart |
| | | 428.9 | failure, Acute on chronic systolic heart failure, Diastolic heart failure, unspecified, Acute |
| | | | diastolic heart failure, Chronic diastolic heart failure, Acute on chronic diastolic heart |
| | | | failure, Combined systolic and diastolic heart failure, unspecified, Acute combined systolic |
| | | | and diastolic heart failure, Chronic combined systolic and diastolic heart failure, Acute on |
| | | | chronic combined systolic and diastolic heart failure, Heart failure, unspecified |
| Heart | ICD10 | I09.81, I11.0, | Rheumatic Heart Failure, Hypertensive Heart Disease With Heart Failure, Hypertensive Heart And |
| Failure | | I13.0, I13.2, | Chronic Kidney Disease With Heart Failure And Stage 1 Through Stage 4 Chronic Kidney Disease, Or |
| | | I50.1, I50.20, | Unspecified Chronic Kidney Disease, Hypertensive Heart And Chronic Kidney Disease With Heart |
| | | I50.21, I50.22, | Failure And With Stage 5 Chronic Kidney Disease, Or End Stage Renal Disease, Left Ventricular |
| | | I50.23, I50.30, | Failure, Unspecified Systolic (Congestive) Heart Failure, Acute Systolic (Congestive) Heart Failure, |
| | | I50.31, I50.32, | Chronic Systolic (Congestive) Heart Failure, Acute On Chronic Systolic (Congestive) Heart Failure, |
| | | I50.33, I50.40, | Unspecified Diastolic (Congestive) Heart Failure, Acute Diastolic (Congestive) Heart Failure, Chronic |
| | | I50.41, I50.42, | |
| | | I50.43, I50.9, | |
| | | I97.130, I97.131 | |
| Myocardial | ICD9 | 410, 410.01, | Diastolic (Congestive) Heart Failure, Acute On Chronic Diastolic (Congestive) Heart Failure, |
| Infarction | | 410.02, 410.1, | Unspecified Combined Systolic (Congestive) And Diastolic (Congestive) Heart Failure, Acute Combined |
| | | 410.11, 410.12, | Systolic (Congestive) And Diastolic (Congestive) Heart Failure, Chronic Combined Systolic |
| | | 410.2, 410.21, | (Congestive) And Diastolic (Congestive) Heart Failure, Acute On Chronic Combined Systolic |
| | | 410.22, 410.3, | (Congestive) And Diastolic (Congestive) Heart Failure, Heart Failure, Unspecified, Postprocedural |
| | | 410.31, 410.32, | heart failure following cardiac surgery, Postprocedural heart failure following other surgery |
| | | 410.4, 410.41, | Acute myocardial infarction of anterolateral wall, episode of care unspecified, Acute myocardial |
| | | 410.42, 410.5, | infarction of anterolateral wall, initial episode of care, Acute myocardial infarction of |
| | | 410.51, 410.52, | anterolateral wall, subsequent episode of care , Acute myocardial infarction of other anterior wall, |
| | | 410.6, 410.61, | episode of care unspecified, Acute myocardial infarction of other anterior wall, initial episode of |
| | | 410.62, 410.7, | care, Acute myocardial infarction of other anterior wall, subsequent episode of care, Acute |
| | | 410.71, 410.72, | myocardial infarction of inferolateral wall, episode of care unspecified, Acute myocardial |
| | | 410.8, 410.81, | infarction of inferolateral wall, initial episode of care, Acute myocardial infarction of |
| | | 410.82, 410.9, | inferolateral wall, subsequent episode of care, Acute myocardial infarction of inferoposterior |
| | | 410.91, 410.92, | wall, episode of care unspecified, Acute myocardial infarction of inferoposterior wall, initial |
| | | 412, 429.79 | episode of care, Acute myocardial infarction of inferoposterior wall, subsequent episode of care, |
| | | | Acute myocardial infarction of other inferior wall, episode of care unspecified, Acute myocardial |
| | | | infarction of other inferior wall, initial episode of care, Acute myocardial infarction of other |
| | | | inferior wall, subsequent episode of care, Acute myocardial infarction of other lateral wall, |
| | | | episode of care unspecified, Acute myocardial infarction of other lateral wall, initial episode |
| | | | of care, Acute myocardial infarction of other lateral wall, subsequent episode of care, True |
| | | | posterior wall infarction, episode of care unspecified, True posterior wall infarction, initial |
| | | | episode of care, True posterior wall infarction, subsequent episode of care, Subendocardial |
| | | | infarction, episode of care unspecified, Subendocardial infarction, initial episode of care, |
| | | | Subendocardial infarction, subsequent episode of care, Acute myocardial infarction of other |
| | | | specified sites, episode of care unspecified, Acute myocardial infarction of other specified |
| | | | sites, initial episode of care, Acute myocardial infarction of other specified sites, subsequent |
| | | | episode of care, Acute myocardial infarction of unspecified site, episode of care unspecified, |
| | | | Acute myocardial infarction of unspecified site, initial episode of care, Acute myocardial |
| | | | infarction of unspecified site, subsequent episode of care, Old myocardial infarction , Certain |
| | | | sequelae of myocardial infarction, not elsewhere classified, other |
| Myocardial | ICD10 | I21.01, I21.02, | ST elevation (STEMI) myocardial infarction involving left main coronary artery, ST elevation |
| Infarction | | I21.09, I21.11, | (STEMI) myocardial infarction involving left anterior descending coronary artery, ST elevation |
| | | I21.19, I21.21, | (STEMI) myocardial infarction involving other coronary artery of anterior wall, ST elevation |
| | | I21.29, I21.3, | (STEMI) myocardial infarction involving right coronary artery, ST elevation (STEMI) myocardial |
| | | I21.4, I22.0, | infarction involving other coronary artery of inferior wall, ST elevation (STEMI) myocardial |
| | | I22.1, I22.2. | infarction involving left circumflex coronary artery, ST elevation (STEMI) myocardial infarction |
| | | I22.8, I22.9, | involving other sites, ST elevation (STEMI) myocardial infarction of unspecified site , Non-ST |
| | | I23.0, I23.1, | elevation (NSTEMI) myocardial infarction , Subsequent ST elevation (STEMI) myocardial infarction |
| | | I23.2, I23.3, | of anterior wall, Subsequent ST elevation (STEMI) myocardial infarction of inferior wall, |
| | | I23.4, I23.5, | Subsequent non-ST elevation (NSTEMI) myocardial infarction , Subsequent ST elevation (STEMI) |
| | | I23.6, I23.7, | myocardial infarction of other sites, Subsequent ST elevation (STEMI) myocardial infarction of |
| | | I23.8, I24.1, | unspecified site, Hemopericardium as current complication following acute myocardial infarction, |
| | | I25.2 | Atrial septal defect as current complication following acute myocardial infarction, Ventricular |
| | | | septal defect as current complication following acute myocardial infarction, Rupture of cardiac |
| | | | wall without hemopericardium as current complication following acute myocardial infarction, |
| | | | Rupture of chordae tendineae as current complication following acute myocardial infarction, |
| | | | Rupture of papillary muscle as current complication following acute myocardial infarction, |
| | | | Thrombosis of atrium, auricular appendage, and ventricle as current complications following |
| | | | acute myocardial infarction, Postinfarction angina, Other current complications following acute |
| | | | myocardial infarction, Dressler's syndrome, Old myocardial infarction |

Atrial fibrillation (AF) defined as ECG diagnosis of AF, ≥1 inpatient diagnosis code, ≥1 procedural code, or ≥2 codes of any type MYOCARDIAL INFARCTION DEFINED AS ≥2 CODES FROM ANY SETTING.
HEART FAILURE DEFINED AS ≥1 INPATIENT DIAGNOSIS CODE

### Statistical Analysis

Applicants tested three approaches for estimating 12-lead ECG-derived \dot{V}O_{2 PEAK}. First, Applicants fit a penalized regression model in which they regressed \dot{V}O_{2 PEAK} on age, sex, BMI, and CPET modality (e.g., bike, treadmill, or rowing ergometer), which we refer to as the “Basic” model. Applicants fit a second model that included age, sex, BMI, and CPET modality, and measured ECG intervals (i.e., ventricular rate, PR interval, QRS duration, and QT interval), which Applicants refer to as “Basic+ECG Parameters.” BMI was included given previous evidence suggesting primary effects of BMI on deep learned ECG representations. Testing modality was included given well-established data on the effect of exercise modality on measured \dot{V}O_{2 PEAK}. Third, Applicants developed a deep learning-based model to predict CPET-derived \dot{V}O_{2 PEAK}, which Applicants refer to as “Deep ECG-\dot{V}O₂.” To develop Deep ECG-\dot{V}O₂, Applicants used an existing deep learning model that creates 320-dimensional embeddings of resting 12-lead ECGs (Patient Contrastive Learning of Representations, PCLR). PCLR is a pre-trained, publicly available convolutional neural network that creates numerical representations of ECG waveforms, which emphasize differences between ECGs from different individuals and similarities between ECGs from the same individual. PCLR representations have been shown to perform well when included in penalized linear models.

For each model, Applicants tested alternative penalization methods (i.e., Ridge, Elastic Net, and Least Absolute Shrinkage and Selection Operator [Lasso]) in the validation set and varied the value of the penalty term using 10-fold cross-validation. Applicants then selected the penalization method and penalty term that minimized the average error across the 10 folds. A Lasso penalty maximized performance for the Basic and Basic+ECG Parameters models, while an Elastic Net penalty maximized performance for the Deep ECG-\dot{V}O₂ model. Each model was then applied to a holdout test set comprising roughly 20% of the study sample in estimate \dot{V}O_{2 PEAK}.

Models were assessed by measuring the Pearson correlation and mean absolute error (MAE) against CPET-derived \dot{V}O_{2 PEAK}. Model metrics were compared using 1,000-iteration bootstrapping. Correlation was depicted using scatterplots. Agreement was assessed by inspecting Bland-Altman plots, where an accurate model has y-axis values close to zero, and an unbiased model shows no systematic pattern along the x-axis (i.e., random scatter).

To assess model performance in identification of individuals with markedly impaired CRF, Applicants compared the diagnostic test characteristics of each model for identifying individuals with estimated \dot{V}O_{2 PEAK}<14 mL/kg/min (a clinical threshold used to define substantially impaired CRF). Given absence of a consensus threshold for mildly impaired CRF, Applicants additionally assessed test characteristics at the sample-level 25^{th} percentile of true \dot{V}O_{2 PEAK} (i.e., <20.9 mL/kg/min). Applicants calculated sensitivity, specificity, positive predictive value, and negative predictive value of the three \dot{V}O_{2 PEAK} estimation models in the test set. Applicants then calculated concordance and net reclassification indices (NRI).

Given that Deep ECG-\dot{V}O₂ showed the highest accuracy for estimation of CPET-derived \dot{V}O_{2 PEAK}, this model was selected for additional evaluation. To assess associations between Deep ECG-\dot{V}O₂ and incident clinical outcomes, Applicants inferred CPET-derived \dot{V}O_{2 PEAK} using Deep ECG-\dot{V}O₂ among over 80,000 individuals in C3PO and not included in the training or validation sets. The single ECG closest to the start of follow-up within the three-year window was used to predict \dot{V}O_{2 PEAK}. Since most individuals in the training set had \dot{V}O_{2 PEAK} measured using bicycle exercise (approximately 60%), this modality was assumed at model inference. For longitudinal analyses, person-time ended at the earliest of an outcome event, death, last encounter of any type in the electronic health record, age 90, or the administrative censoring date for C3PO (Aug. 31, 2019). Since death commonly occurred after the last encounter or administrative censoring date, person-time was not censored at these timepoints for the death outcome.

Associations between Deep ECG-\dot{V}O₂ estimated \dot{V}O_{2 PEAK} and incident AF, MI, HF, and all-cause death were assessed using Cox proportional hazards models with Deep ECG-\dot{V}O₂ estimated \dot{V}O_{2 PEAK} as the exposure of interest, with adjustment for age, sex, and BMI. Deep ECG-\dot{V}O₂ estimated \dot{V}O_{2 PEAK} was modeled as a continuous variable, per quartile, and at pre-specified thresholds of a)<14 mL/kg/min (our pre-specified threshold used to define markedly impaired CRF) and b)<25^{th} percentile of Deep ECG-\dot{V}O₂ estimated \dot{V}O_{2 PEAK} observed in the disease analysis sample (i.e., <25.1 mL/kg/min). The proportional hazards assumption was assessed by inspecting Schoenfeld residuals. Substantial deviations from proportional hazards (observed only for covariates) were modeled using interaction terms including strata of person-time. Death was modeled as a censoring event in the primary analyses, but in secondary analyses, death was modeled as a competing risk. Kaplan-Meier curves were used to plot cumulative disease incidence stratified by Deep ECG-\dot{V}O₂ estimated \dot{V}O_{2 PEAK}<14 mL/kg/min versus 14 mL/kg/min (and secondarily <25^{th} percentile versus >25^{th} percentile). Adjusted curves were generated by plotting predicted risk from stratified Cox models with each disease as the outcome and stratum of Deep ECG-\dot{V}O₂ estimated \dot{V}O_{2 PEAK} as a stratification variable, with adjustment for age, sex, and BMI.

To assess regions of the ECG waveform having the greatest effect on Deep ECG-\dot{V}O₂ estimations, Applicants plotted the median ECG waveforms for individuals with Deep ECG-\dot{V}O₂ estimated \dot{V}O_{2 PEAK} at or above the 90^{th} percentile (i.e., ≥48 mL/kg/min) versus individuals with Deep ECG-\dot{V}O₂ estimated \dot{V}O_{2 PEAK} below the 10^{th} percentile (i.e., <16 mL/kg/min) in the holdout set independent of model training. As performed previously, to generate median waveforms, the median voltage across all individuals within the respective stratum is plotted for each time bin included in a single cardiac cycle.

All analyses were performed using Python v3.88 and R v3.69 (packages ‘data.table’, ‘stringr’, ‘glmnet’, ‘pROC’, ‘nricens’, ‘cmprsk’). Two-sided p values<0.05 were considered to indicate statistical significance.

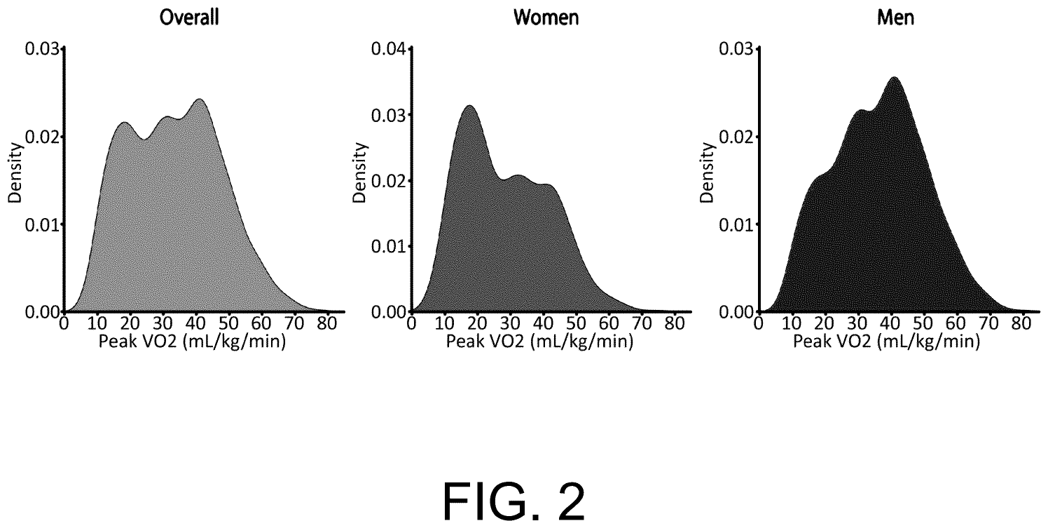

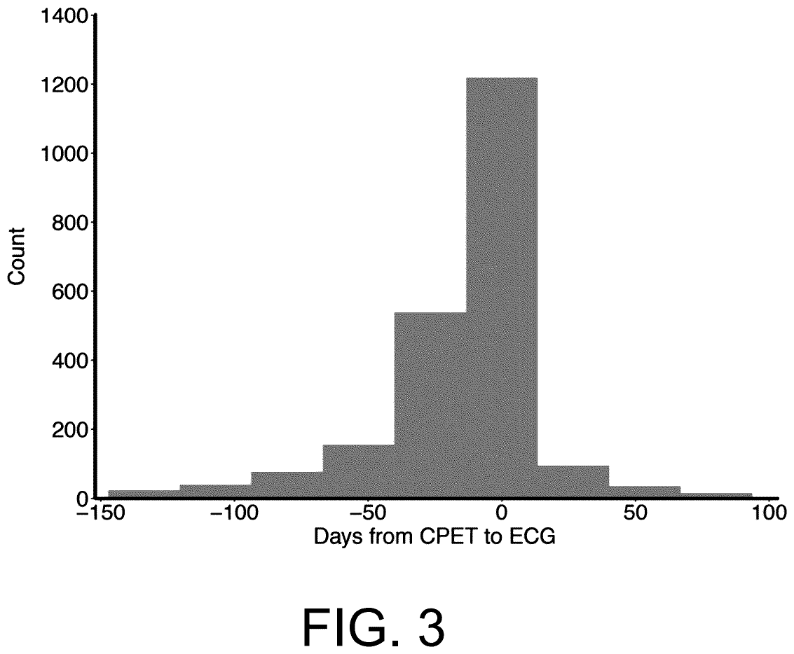

After excluding individuals without resting 12-lead ECGs available within one year of the CPET and those with missing demographic, exercise or anthropometric data, there were a total of 2,339 individuals included in the model derivation set (FIG. 1). Individuals included had a mean age of 46 years (standard deviation 19 years), and 37% were women. The mean \dot{V}O_{2 PEAK} on CPET was 33.6 mL/kg/min (standard deviation 14.4) overall, 36.4 mL/kg/min (14.2) for men, and 28.8 mL/kg/min (13.5) for women (FIG. 2). A total of 198 individuals in the analysis set (8.5%) had true \dot{V}O_{2 PEAK}<14 mL/kg/min. The ECG used for inference was performed a median of 7 days (quartile-1: 27 days, quartile-3: 0 days) before CPET (FIG. 3). The sample was divided into training/validation (n=1,891) and test (n=448) sets. Detailed characteristics are shown in Table 2.

TABLE 2
Sample characteristics

| | Training set | | | |
|---|---|---|---|---|
| | (n = 1,410) | Validation set | Test set | Disease set |
| | Mean ± SD or N (%) | (n = 481) | (n = 448) | (n = 84,718) |
| Age, years | 45.4 ± 19.2 | 46.1 ± 19.5 | 45.7 ± 19.8 | 55.3 ± 16.3 |
| Female | 536 (38.0%) | 167 (34.7%) | 173 (38.6%) | 38,284 (45.2%) |
| Body mass index, kg/m² | 25.9 ± 4.9 | 26.2 ± 4.7 | 26.2 ± 5.1 | 28.8 ± 6.6 |
| Clinical Characteristics | | | | |
| Hypertension | 369 (26.3%) | 112 (23.3%) | 107 (24.0%) | 49,508 (58.4%) |
| Diabetes Mellitus | 55 (3.9%) | 20 (4.2%) | 16 (3.6%) | 16,893 (19.9%) |
| Hyperlipidemia | 395 (28.4%) | 136 (28.6%) | 128 (28.9%) | 46,044 (54.3%) |
| Myocardial Infarction | 86 (6.1%) | 28 (5.9%) | 22 (4.9%) | 7,990 (9.4%) |
| Heart failure | 56 (4.0%) | 18 (3.7%) | 16 (3.6%) | 6,456 (7.6%) |
| Atrial Fibrillation | 136 (9.7%) | 47 (9.8%) | 42 (9.4%) | 9,885 (11.7%) |
| CPET modality | | | | |
| Bicycle | 872 (61.8%) | 292 (60.7%) | 285 (63.6%) | — |
| Treadmill | 494 (35.0%) | 170 (35.3%) | 144 (32.1%) | — |
| Rower | 44 (3.1%) | 19 (4.0%) | 19 (4.2%) | — |
| ECG characteristics | | | | |
| Ventricular rate, bpm | 65.8 ± 13.7 | 66.3 ± 14.6 | 66.6 ± 14.0 | — |
| PR interval, ms | 163.3 ± 40.9 | 161.9 ± 34.9 | 164.9 ± 53.0 | — |
| QRS interval, ms | 97.2 ± 21.4 | 96.8 ± 20.9 | 97.9 ± 21.9 | — |
| QT interval, ms | 409.7 ± 37.4 | 406.9 ± 38.4 | 410.2 ± 37.4 | — |
| True \dot{V}O_{2 PEAK}, | 33.6 ± 14.3 | 34.0 ± 14.3 | 32.9 ± 14.4 | — |
| mL/kg/min | | | | |
| Deep ECG-\dot{V}O2 | 33.6 ± 11.7 | 33.9 ± 12.1 | 33.5 ± 12.1 | 30.3 ± 8.9 |
| estimated \dot{V}O_{2 PEAK}, | | | | |
| mL/kg/min | | | | |

CPET = Cardiopulmonary Exercise Test;
ECG = electrocardiogram;
SD = standard deviation

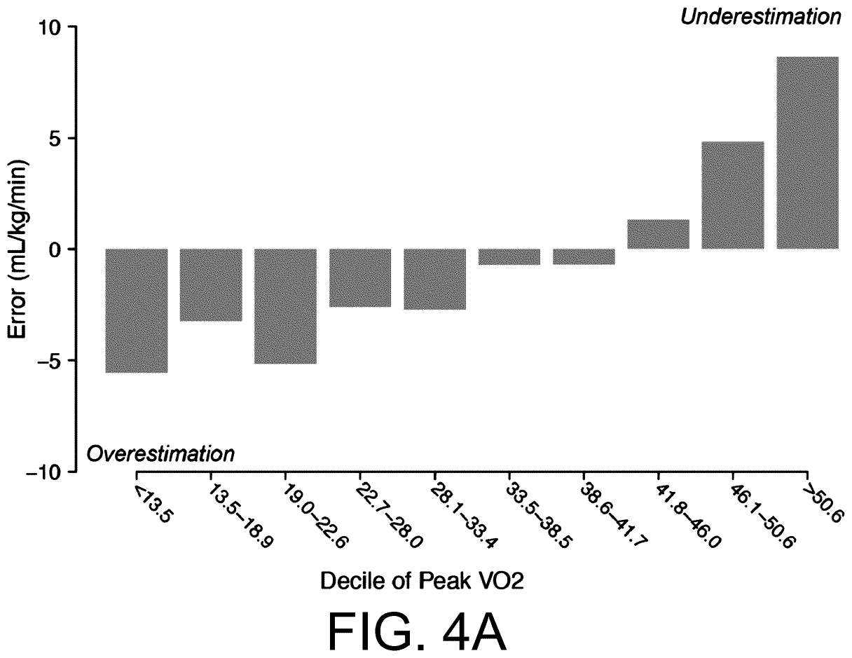

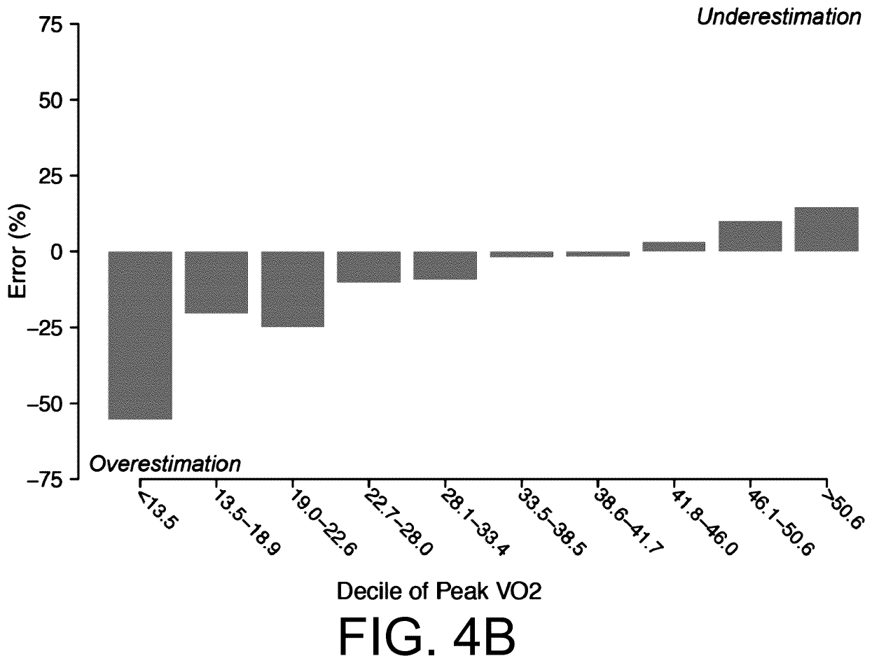

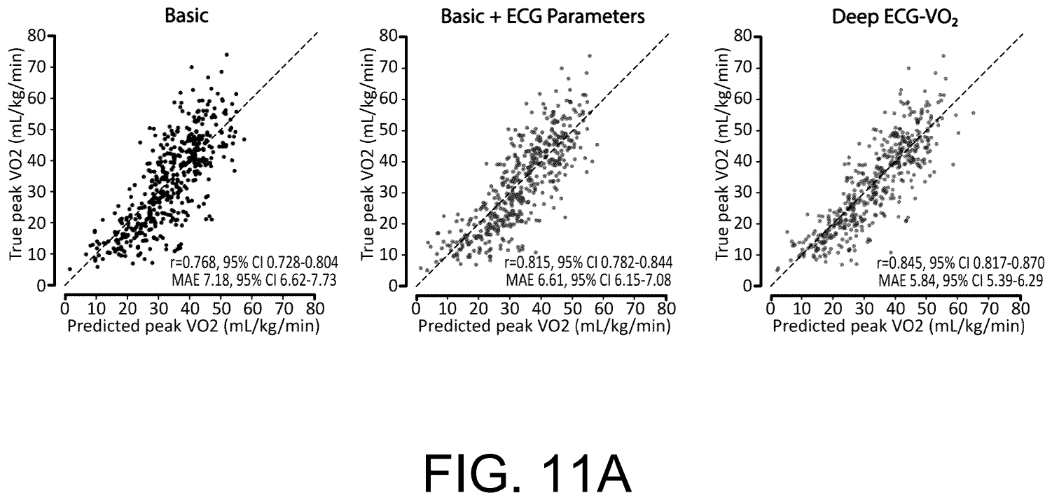

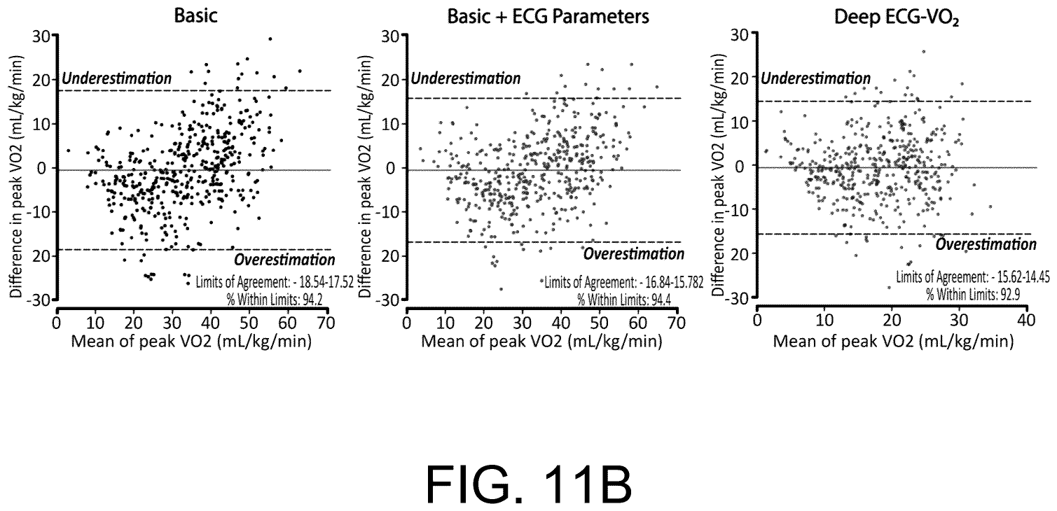

When evaluated in the test set, estimated \dot{V}O_{2 PEAK} using each of the three models correlated with true CPET-derived \dot{V}O_{2 PEAK} (FIG. 11A, 11B). Correlation with true \dot{V}O_{2 PEAK} was lowest with the Basic model (r=0.768, 95% CI 0.728-0.804, MAE 7.18 mL/kg/min, 95% CI 6.62-7.73) and incrementally greater with the Basic+ECG Parameters model (r=0.815, 95% CI 0.782-0.844, MAE 6.61 mL/kg/min, 95% CI 6.15-7.08). Correlation was highest and error was lowest using Deep ECG-\dot{V}O₂ (r=0.845, 95% CI 0.817-0.870 and MAE 5.84 mL/kg/min, 95% CI 5.39-6.29; p<0.01 for comparison of correlation and MAE to the other two models), with average error of Deep ECG-\dot{V}O₂ representing roughly one-third the standard deviation of true \dot{V}O_{2 PEAK} (MAE 5.84 mL/kg/min, 95% CI 5.39-6.29). Estimation error using Deep ECG-\dot{V}O₂ was generally greatest at the extremes of true \dot{V}O_{2 PEAK} (FIGS. 4A and 4B).

Agreement between model-estimated and observed \dot{V}O_{2 PEAK} values was similarly most favorable with Deep ECG-\dot{V}O₂ (95% limits of agreement −15.62 to 14.45) as compared to either Basic (95% limits of agreement −18.54 to 17.52) or Basic+ECG Parameters (95% limits of agreement −16.84 to 15.78). Both the Basic and Basic+ECG Parameters models also demonstrated evidence of bias, in which model estimates were conservative (i.e., overestimation of low true values, and underestimation of high true values). No such pattern was observed using Deep ECG-\dot{V}O₂. Bland-Altman plots are shown in FIGS. 11A and 11B.

Test characteristics of the three models showed a similar pattern. Each of the models discriminated impaired \dot{V}O_{2 PEAK} at the 25^{th} percentile threshold of <20.9 mL/kg/min. Discrimination was lowest using the Basic model (0.723, 95% CI 0.675-0.771), intermediate using Basic+ECG Parameters (c-statistic 0.756, 95% CI 0.709-0.804, p=0.17), and greatest with Deep ECG-\dot{V}O₂ (c-statistic 0.788, 95% CI 0.740-0.834, p<0.01 vs. Basic and p=0.17 vs. Basic+ECG Parameters). Similar results were observed at the \dot{V}O_{2 PEAK}<14 mL/kg/min threshold (Basic 0.646, 95% CI 0.579-0.713; Basic+ECG Parameters 0.662, 95% CI 0.592-0.732; Deep ECG-\dot{V}O₂ 0.675, 95% CI 0.604-0.746; p=0.41 vs. Basic and p=0.69 vs. Basic+ECG Parameters). In general, Deep ECG-\dot{V}O₂ demonstrated greater sensitivity while maintaining high specificity for impaired \dot{V}O_{2 PEAK}. Detailed test characteristics at each threshold are shown in Table 3.

TABLE 3
Diagnostic test characteristics for impaired \dot{V}O_{2 PEAK} in the independent test sets

| | | | | | | |
|---|---|---|---|---|---|---|
| | | | Positive | Negative | | |
| | Sensitivity* | Specificity | predictive | predictive | c-statistic | |
| Model | (95% CI) | (95% CI) | value (95% CI) | value (95% CI) | (95% CI) | p* |

<20.9 mL/kg/min (25^{th} percentile)

| | | | | | | |
|---|---|---|---|---|---|---|
| Basic | 47 (38-57) | 97 (95-99) | 85 (74-93) | 85 (81-88) | 0.723 (0.675-0.771) | <0.01 |
| Basic + ECG | 54 (44-63) | 98 (95-99) | 88 (78-95) | 87 (83-90) | 0.756 (0.709-0.804) | 0.17 |
| Deep ECG-\dot{V}O₂ | 60 (50-69) | 97 (95-99) | 88 (78-94) | 88 (84-91) | 0.788 (0.740-0.834) | referent |

<14 mL/kg/min

| | | | | | | |
|---|---|---|---|---|---|---|
| Basic | 30 (18-46) | 99 (97-100) | 74 (49-91) | 93 (90-95) | 0.646 (0.579-0.713) | 10.41 |
| Basic + ECG | 35 (21-50) | 98 (95-99) | 62 (41-80) | 93 (90-95) | 0.662 (0.592-0.732) | 0.69 |
| Deep ECG-\dot{V}O₂ | 37 (23-52) | 98 (96-99) | 68 (46-85) | 93 (90-95) | 0.675 (0.604-0.746) | referent |

*P-value for pairwise comparison with Deep ECG-\dot{V}O₂

Net reclassification using Deep ECG-\dot{V}O₂ was favorable at the 25^{th} percentile threshold of <20.9 mL/kg/min as compared to Basic (NRI 0.13, 95% CI 0.043-0.22) or Basic+ECG Parameters (0.061, 95% CI −0.025-0.16), driven primarily by higher case detection (NRI+12.7%, 95% CI 5.1%-22.0% versus Basic; 6.1%, 95% CI −2.5%-15.9% versus Basic+ECG Parameters). Findings were similar at the \dot{V}O_{2 PEAK}<14 mL/kg/min threshold (NRI 0.058, 95% CI −0.072-0.19 vs Basic; 0.027, 95% CI −0.063-0.15 vs Basic+ECG Parameters), again driven by case detection (NRI+ 6.5%, 95% CI −6.7%-20.0% versus Basic; 2.7%, 95% CI −6.3%-15.4% versus Basic+ECG Parameters. Details of net reclassification analyses are shown in Tables 4 and 5.

TABLE 4
Reclassification analysis comparing Deep ECG-\dot{V}O₂ to comparison models
for identifying \dot{V}O_{2 PEAK} <20.9 mL/kg/min (25^{th} percentile)

| | | Deep ECG-\dot{V}O₂ | | | Deep ECG-\dot{V}O₂ | |
|---|---|---|---|---|---|---|
| | Basic | No | Yes | Basic + ECG | No | Yes |
| Impaired* | No | 37 | 21 | No | 36 | 15 |
| | Yes | 7 | 45 | Yes | 8 | 51 |
| Not | No | 325 | 4 | No | 327 | 3 |
| Impaired* | Yes | 4 | 5 | Yes | 2 | 6 |

| | | | |
|---|---|---|---|
| NRI+ | 12.7% | NRI+ | 6.1% |
| | (−5.1% to 20.0%) | | (−2.5% to 15.9%) |
| NRI− | −0.8% | NRI− | 0.5% |
| | (−2.2% to 0.7%) | | (−1.2% to 2.0%) |
| NRI | 0.13 | NRI | 0.061 |
| | (0.043 to 0.22) | | (−0.025 to 0.16) |

Bold: Appropriate reclassification
Italics: Inappropriate reclassification
*Impairment defined as \dot{V}O_{2 PEAK} <20.9 mL/kg/min (25^{th} percentile)
NRI = net reclassification index
NRI+ = case reclassification
NRI− = non-case reclassification

TABLE 5
Reclassification analysis comparing Deep ECG-\dot{V}O₂ to comparison
models for identifying \dot{V}O_{2 PEAK} <14 mL/kg/min

| | | Deep ECG-\dot{V}O₂ | | | Deep ECG-\dot{V}O₂ | |
|---|---|---|---|---|---|---|
| | Basic | No | Yes | Basic + ECG | No | Yes |
| Impaired* | No | 25 | 7 | No | 25 | 5 |
| | Yes | 4 | 10 | Yes | 4 | 12 |
| Not | No | 392 | 5 | No | 388 | 4 |
| Impaired* | Yes | 2 | 3 | Yes | 6 | 4 |

| | | | |
|---|---|---|---|
| NRI+ | 6.5% | NRI+ | 2.7% |
| | (−6.7% to 20.0%) | | (−6.3% to 15.4%) |
| NRI− | −0.8% | NRI− | 0.5% |
| | (−2.2% to 0.7%) | | (−1.2% to 2.0%) |
| NRI | 0.058 | NRI | 0.027 |
| | (−0.072 to 0.19) | | (−0.063 to 0.15) |

Bold: Appropriate reclassification
Italics: Inappropriate reclassification
*Impairment defined as \dot{V}O_{2 PEAK} <14 mL/kg/min
NRI = net reclassification index
NRI+ = case reclassification
NRI− = non-case reclassification

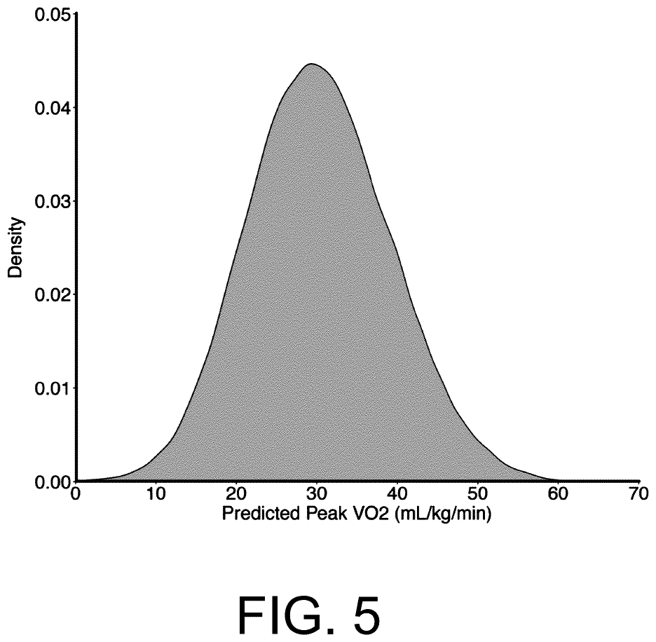

Applicants then used Deep ECG-\dot{V}O₂ to estimate \dot{V}O_{2 PEAK} within 84,718 individuals in an ambulatory sample who did not undergo CPET and were not included in model training. The mean Deep ECG-\dot{V}O₂ estimated \dot{V}O_{2 PEAK} was 30.3 mL/kg/min (standard deviation 8.9), and a total of 2,271 individuals (2.7%) had estimated \dot{V}O_{2 PEAK}<14 mL/kg/min (FIG. 5). Deep ECG-\dot{V}O₂ estimated \dot{V}O_{2 PEAK}<14 mL/kg/min was associated with increased risk of AF (hazard ratio [HR] 1.36, 95% CI 1.21-1.54), MI (HR 1.21, 95% CI 1.02-1.45), HF (HR 1.67, 95% CI 1.49-1.88), and all-cause death (HR 1.84, 95% CI 1.68-2.03) (Table 6). Similar associations were observed when assessing Deep ECG-\dot{V}O₂ estimated \dot{V}O_{2 PEAK} at the sample-level 25^{th} percentile threshold of 25.1 mL/kg/min, per quartile of estimated \dot{V}O_{2 PEAK}, and using estimated \dot{V}O_{2 PEAK} as a continuous variable (Table 6).

TABLE 6
Associations between Deep ECG-VO₂ estimated VO_{2 PEAK} and incident disease

| | | | Hazard ratio for exposure | | | |
|---|---|---|---|---|---|---|
| | | | (95% CI)* | | | |
| | | | Estimated | Estimated | | |
| | | | \dot{V}O_{2 PEAK} | \dot{V}O_{2 PEAK} | Estimated | Estimated |
| | N events/ | Follow-up, | (per 1 SD | (per quartile | \dot{V}O_{2 PEAK} | \dot{V}O_{2 PEAK} |
| | N total^{†} | yrs (Q1, Q3) | decrease) | decrease) | <14 mL/kg/min | <25^{th} percentile^{‡} |
| Atrial | 7515/74778 | 5.9 (2.4, 10.9) | 1.44 (1.38-1.50) | 1.26 (1.22-1.30) | 1.36 (1.21-1.54) | 1.43 (1.35-1.52) |
| fibrillation | | | | | | |
| Myocardial | 3522/76674 | 5.9 (2.4, 10.9) | 1.63 (1.54-1.73) | 1.37 (1.31-1.44) | 1.21 (1.02-1.45) | 1.57 (1.44-1.72) |
| infarction | | | | | | |
| Heart | 5901/78207 | 6.0 (2.4, 11.0) | 2.05 (1.95-2.14) | 1.58 (1.53-1.64) | 1.67 (1.49-1.88) | 1.98 (1.86-2.12) |
| failure | | | | | | |
| All-cause | 9314/84718 | 6.3 (2.6, 11.3) | 2.03 (1.96-2.10) | 1.59 (1.54-1.63) | 1.84 (1.68-2.03) | 2.06 (1.95-2.17) |
| mortality | | | | | | |

*All exposures derived using Deep ECG-\dot{V}O₂ (see text). Hazard ratios obtained using Cox proportional hazards models adjusted for age, sex, and body mass index
^{†}Includes individuals without the prevalent condition at the start of cohort follow-up
^{‡}Sample-based 25^{th} percentile of estimated \dot{V}O_{2 PEAK} approximately 24.2 mL/kg/min
SD = standard deviation

TABLE 7
Associations between Deep ECG-\dot{V}O₂ predicted \dot{V}O_{2 PEAK} and incident disease with death as a competing risk

| | | | Hazard ratio for exposure (95% CI)* | | | |
|---|---|---|---|---|---|---|
| | | | Predicted | Predicted | | |
| | | | \dot{V}O_{2 PEAK} | \dot{V}O_{2 PEAK} | Predicted | Predicted |
| | N events/ | Follow-up, | (per 1 SD | (per quartile | \dot{V}O_{2 PEAK} | \dot{V}O_{2 PEAK} |
| | N total^{†} | yrs (Q1, Q3) | decrease) | decrease) | <14 mL/kg/min | <25^{th} percentile^{‡} |
| Atrial | 7515/74778 | 5.9 (2.4, 10.9) | 1.41 (1.37-1.48) | 1.25 (1.21-1.29) | 1.34 (1.19-1.52) | 1.40 (1.32-1.50) |
| fibrillation | | | | | | |
| Myocardial | 3522/76674 | 5.9 (2.4, 10.9) | 1.60 (1.51-1.69) | 1.36 (1.29-1.42) | 1.19 (0.98-1.44) | 1.54 (1.39-1.70) |
| infarction | | | | | | |
| Heart | 5901/78207 | 6.0 (2.4, 11.0) | 2.02 (1.92-2.12) | 1.57 (1.51-1.64) | 1.64 (1.43-1.87) | 1.96 (1.81-2.11) |
| failure | | | | | | |

*All exposures derived using Deep ECG-\dot{V}O₂ (see text). Hazard ratios obtained using subdistribution hazards models adjusted for age, sex, and body mass index and adjusted for death as a competing risk
^{†}Includes individuals without the prevalent condition at the start of cohort follow-up
^{‡}Sample-based 25^{th} percentile of predicted \dot{V}O_{2 PEAK} approximately 24.2 mL/kg/min
SD = standard deviation

Results were also similar when death was modeled as a competing risk (Table 7).

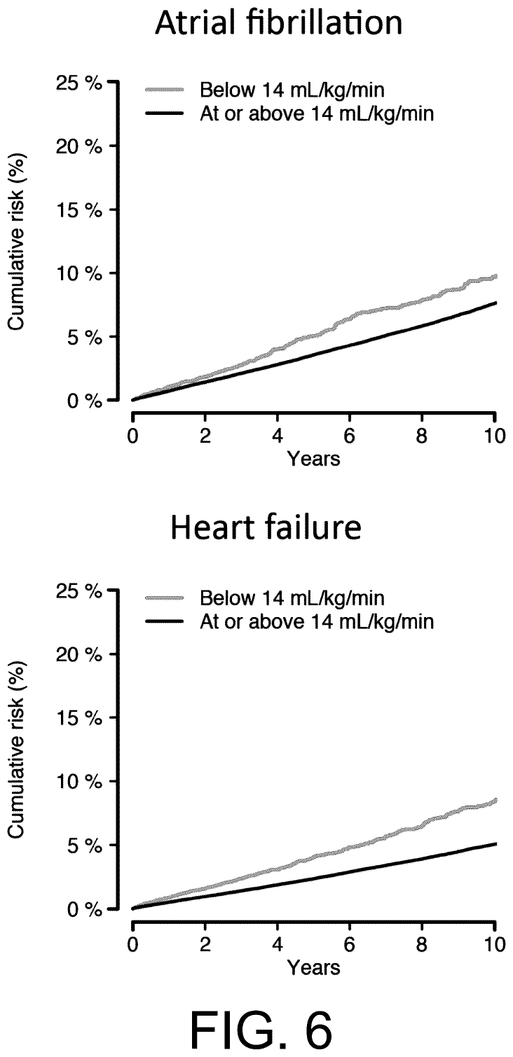

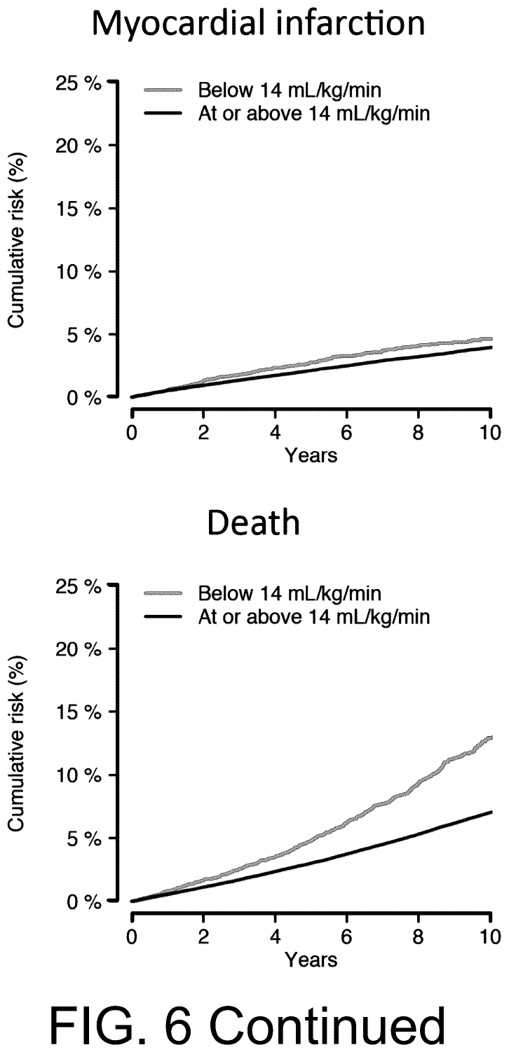

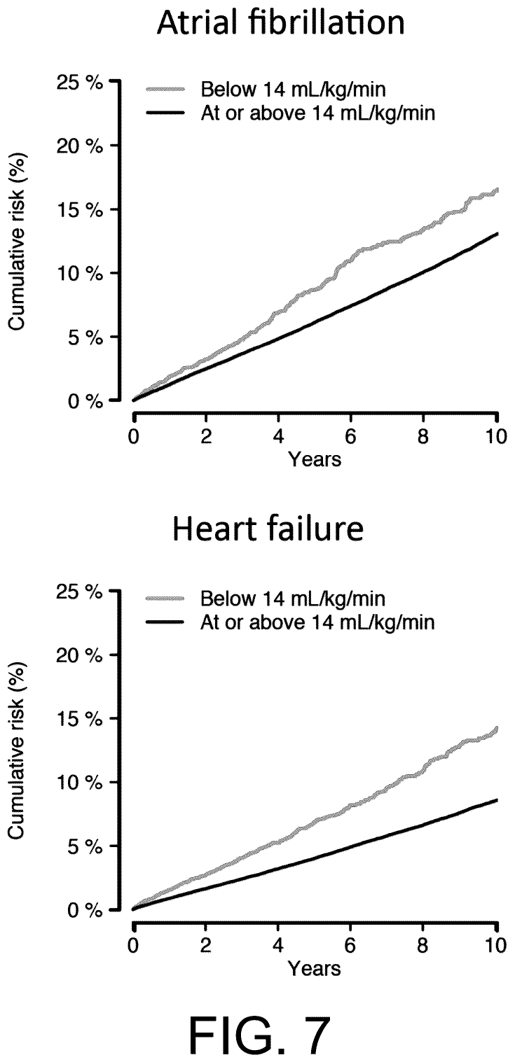

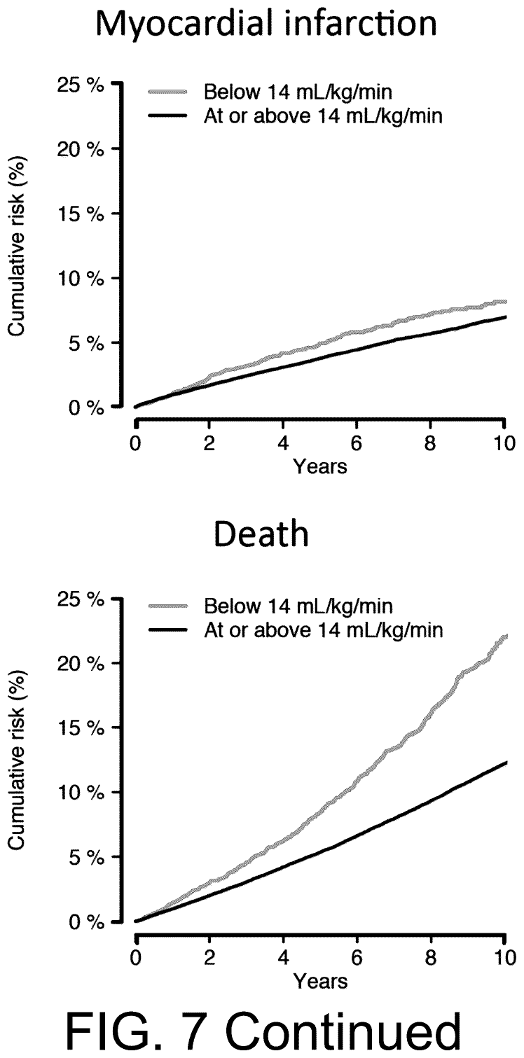

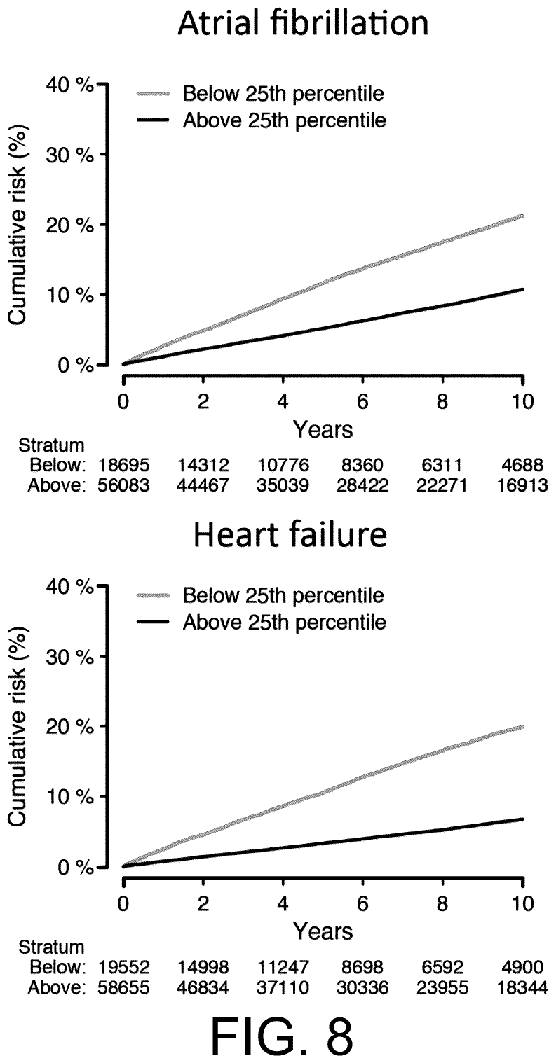

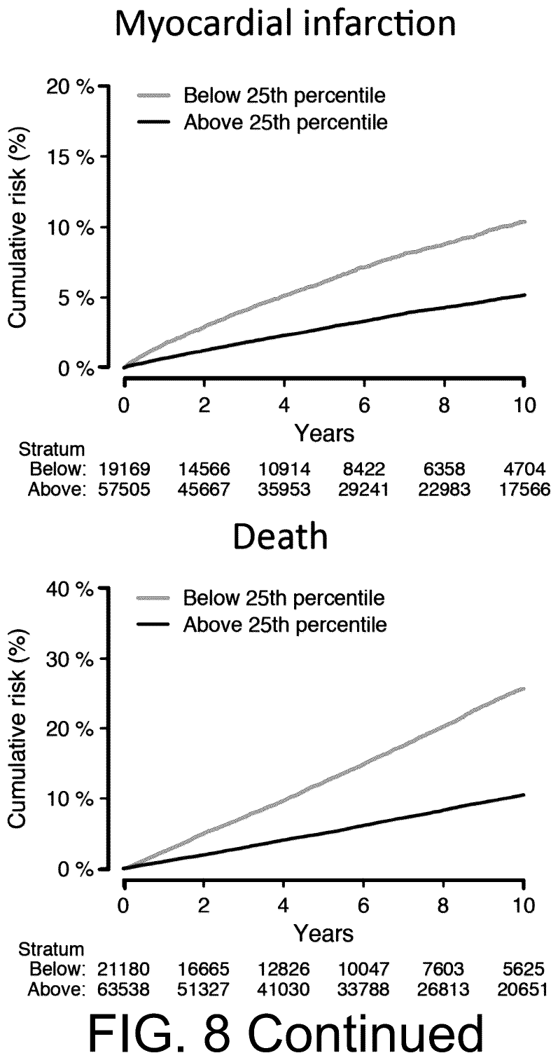

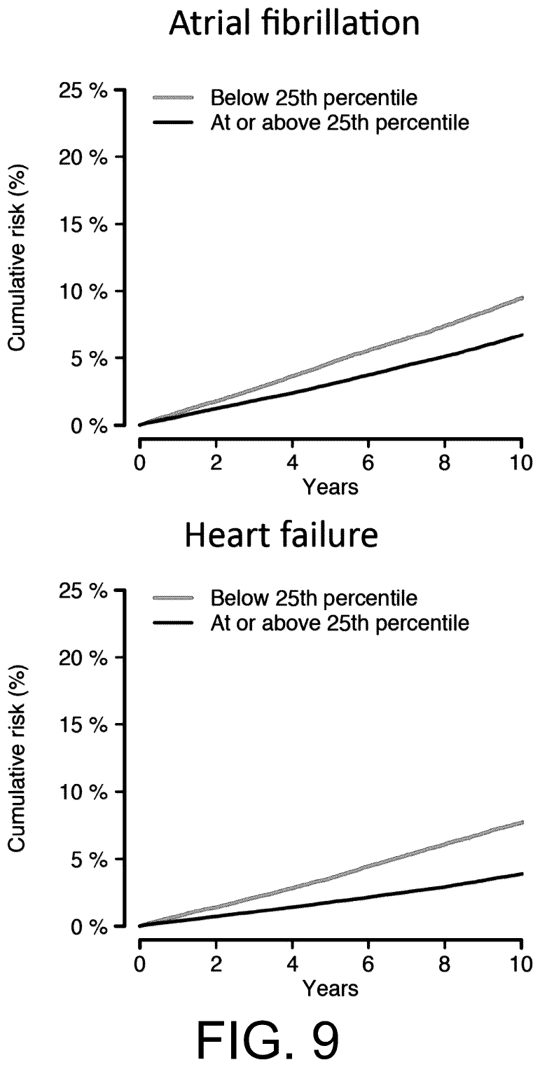

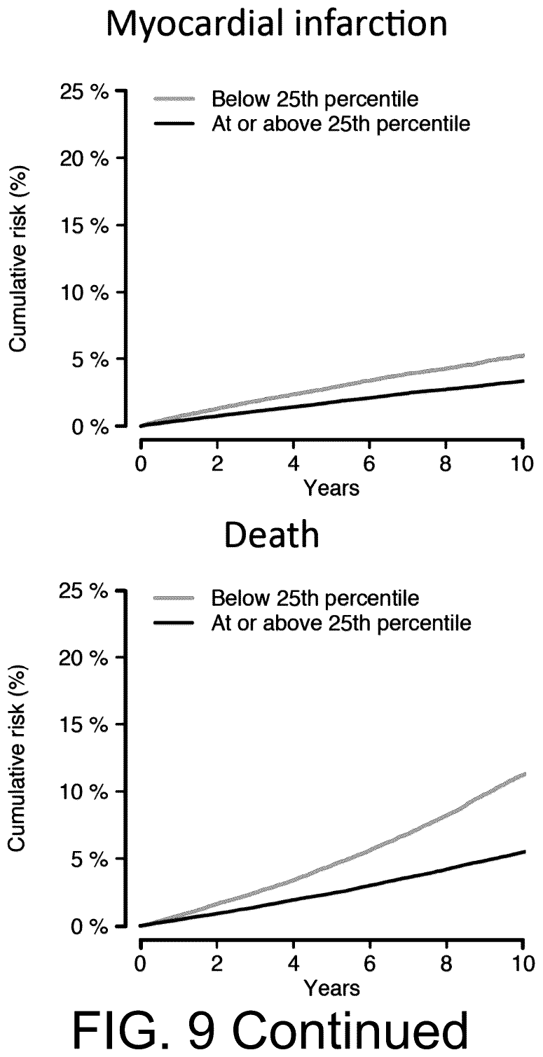

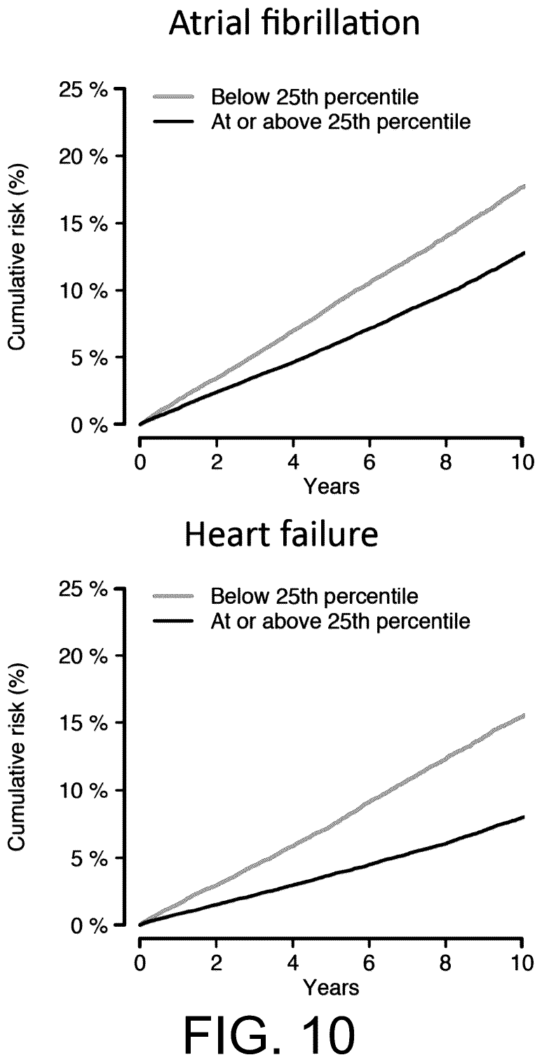

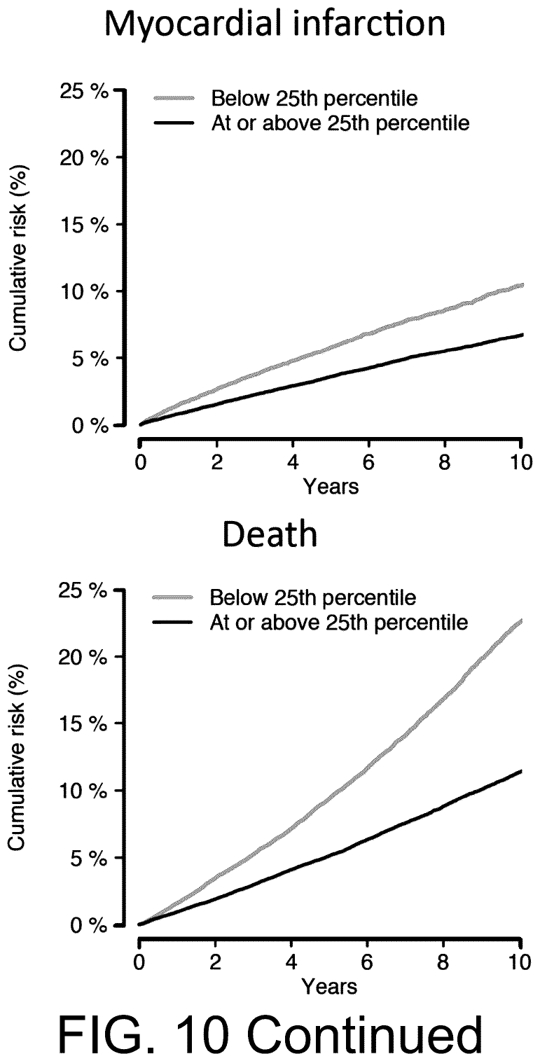

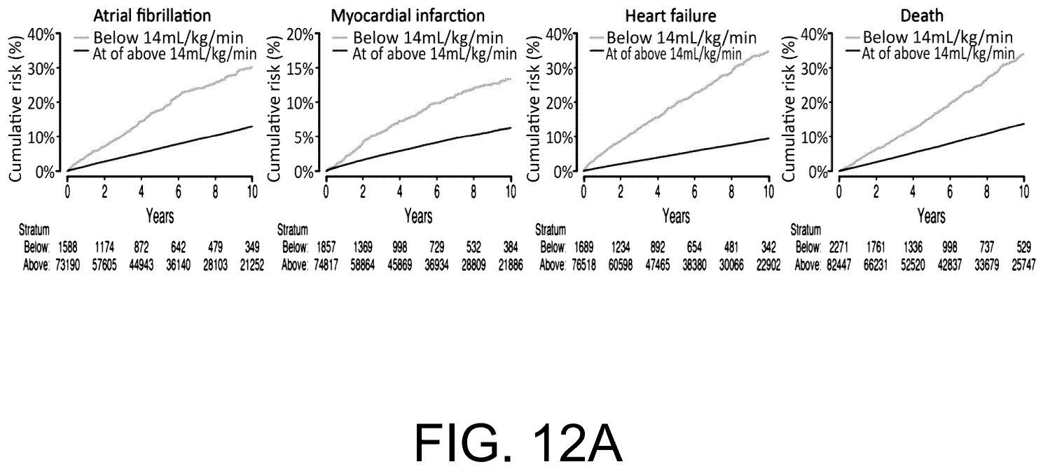

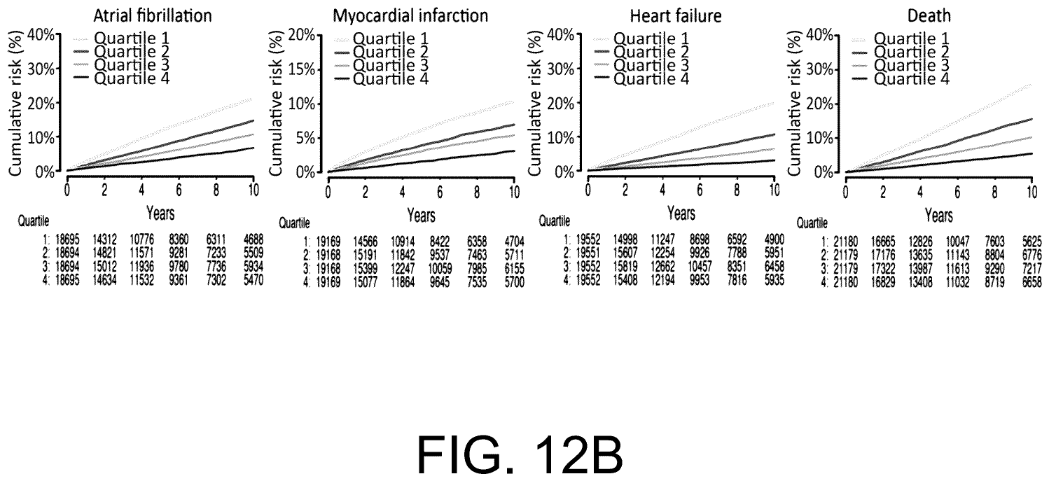

Kaplan-Meier curves demonstrated substantial separation of longitudinal disease incidence stratified at the <14 mL/kg/min threshold as well as with increasing quartile of Deep ECG-\dot{V}O₂ estimated \dot{V}O_{2 PEAK} (FIGS. 12A and 12B). Adjusted curves were similar and are shown in FIGS. 6 and 7). Corresponding crude and adjusted curves stratified at the sample-level 25^{th} percentile threshold of 25.1 mL/kg/min are shown in FIGS. 8-10.

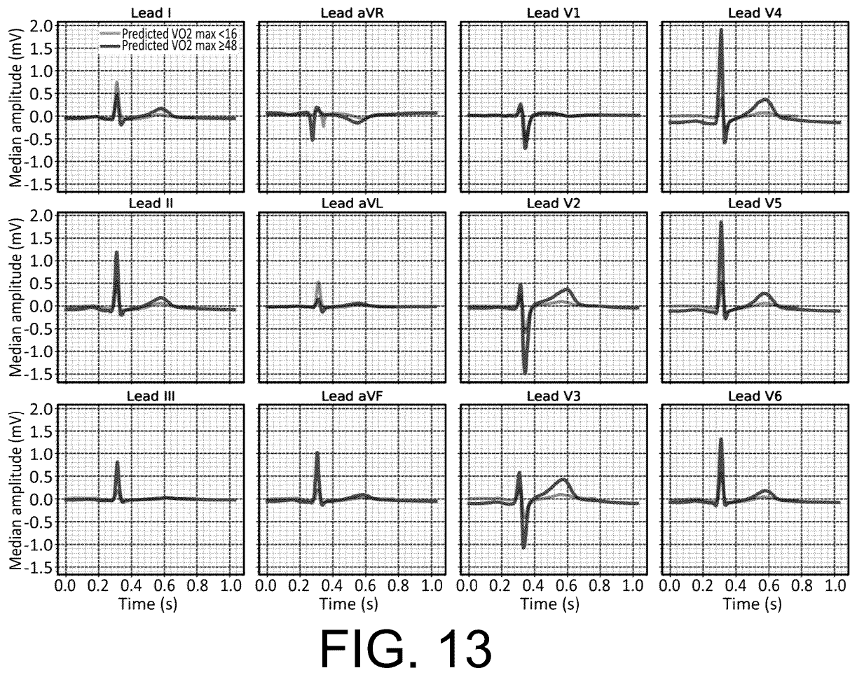

Median waveform analysis demonstrated that individuals with higher Deep ECG-\dot{V}O₂ estimated \dot{V}O_{2 PEAK} appeared to have higher R and S wave amplitudes, especially in the inferior and precordial leads, as well as more prominent ST segments and T waves (FIG. 13).

Applicants present here data on the development and validation of models capable of automated estimation of CPET-derived \dot{V}O_{2 PEAK} using resting 12-lead ECGs. Key findings are as follows. First, ECG-based estimation of \dot{V}O_{2 PEAK} is both feasible and accurate. Applicants demonstrate that this can be performed using models comprising basic clinical factors (age, sex, body size), with and without standard ECG intervals, with reasonable accuracy. Applicants also show, however, that a deep learning approach offered superior accuracy and agreement with less estimation bias, with a correlation of 85% with gold-standard true \dot{V}O_{2 PEAK} and mean absolute error of 5.8 mL/kg/min when assessed in an independent holdout set, with the average error representing only one-third the standard deviation of true \dot{V}O_{2 PEAK}.

Second, consistent with expectations, median waveform analyses suggested that ECG signals relevant for \dot{V}O_{2 PEAK} estimation appear enriched within the R and S wave amplitudes, the ST segment, and T waves.

Third, lower CRF as estimated by Deep ECG-\dot{V}O₂ is a robust predictor of adverse cardiovascular outcomes and higher overall mortality in an independent ambulatory population. Applicants' results collectively suggest that deep learning-encoded ECG data may facilitate accurate and efficient estimation of \dot{V}O_{2 PEAK} as a surrogate for CRF.

A growing body of literature, developed over the past decade, has demonstrated the ability of automated, and often deep learning-based, approaches to extract clinical information latently encoded in the 12-lead resting ECG. Prior work has shown the ability to extract ECG diagnoses and basic characteristics such as age and sex, to classify cardiovascular phenotypes including atrial fibrillation, left ventricular systolic dysfunction, hypertrophic cardiomyopathy, pulmonary arterial hypertension, and cardiac amyloidosis, as well as to predict incident disease such as atrial fibrillation. However, Applicants are unaware of large deep learning studies that examine long term clinical outcomes. Applicants' findings extend this literature by applying a deep learning approach to a distinct phenotype (\dot{V}O_{2 PEAK}) with potent clinical and prognostic value, demonstrating that fitness is latently encoded in the resting ECG and that automated approaches including deep learning can extract and quantify it. Moreover, Applicants showed that estimation of \dot{V}O_{2 PEAK} strongly predicts future cardiovascular outcomes in over 80,000 adults. Notably, \dot{V}O_{2 PEAK} represents a cardiovascular metric that is available only at limited scale, due to constraints on accessibility to specialized exercise testing required for \dot{V}O_{2 PEAK} measurement. Unlike phenotypes that can be assessed in the hundreds of thousands or millions (such as left ventricular systolic function or atrial fibrillation), data on \dot{V}O_{2 PEAK} typically have sample sizes in the hundreds or thousands at best. In the development of Applicants' deep learning model, however, Applicants were able to overcome this methodological challenge by leveraging the pre-trained PCLR model, an approach that can leverage the richness of deep learned representations even when availability of labeled training data is limited.

Data linking CRF to cardiovascular and overall health and correspondingly impaired \dot{V}O_{2 PEAK} to adverse cardiovascular outcomes including heart failure and myocardial infarction typically lack scale for the reasons outlined above. Applicants' results clearly link varying degrees of impairment in \dot{V}O_{2 PEAK} to higher risk of myocardial infarction, heart failure, atrial fibrillation, and overall mortality in a large ambulatory sample. When assessed as a continuous marker and per quartile, higher estimated \dot{V}O_{2 PEAK} was associated with progressively lower disease risk. Therefore, Applicants' ECG-based models, and in particular Deep ECG-\dot{V}O₂, appear to represent an integrative metric of cardiovascular risk that provides relevant prognostic information across multiple specific outcomes and across a wide range of CRY. These findings represent a powerful expansion of the prognostic power of \dot{V}O_{2 PEAK}, as the ability to provide automated extraction of this information from the 12-lead ECG may allow much easier and more widely disseminated access to this important prognostic marker.

Applicants' results suggest that use of Deep ECG-\dot{V}O₂ may be particularly useful for the detection of individuals with impaired CRF, given favorable sensitivity and case reclassification when compared to baseline models. The potential clinical applications of this approach are broad, as accurate \dot{V}O_{2 PEAK} estimation could have powerful implications for varied clinical populations where CRY assessment is prognostically useful but access to a dedicated CPET lab is limited, or exercise is not feasible. Perhaps the most salient of these is in perioperative risk stratification, where there is a robust literature base supporting the ability of CRF to risk stratify both short- and long-term outcomes across a wide range of patient populations and surgical procedures, including both cardiac and non-cardiac surgery. The clinical assessment of CRF is accordingly incorporated into guideline-codified approaches to perioperative risk stratification. Other populations in which easily accessible \dot{V}O_{2 PEAK} data may offer prognostic information include adults with congenital heart disease, cancer survivors, and pregnancy.

More broadly, automated \dot{V}O_{2 PEAK} estimation using standard 12-lead ECG, a procedure performed nearly universally at routine cardiology clinic visits, provides perhaps the best pathway towards fulfilling the American Heart Association's recently stated goal of incorporating CRF as a new ‘clinical vital sign.’ A variety of non-exercise prediction equations have been developed towards estimating CRF, but these are of limited accuracy and can be cumbersome to implement (often incorporating self-assessment of activity or functional status). An ECG-based approach could eliminate these implementation challenges, provide important standardization, and provide an opportunity for continuous improvement with accrual of additional data and model refinement. Accordingly, deployment of Deep ECG-\dot{V}O₂ at scale as a ‘clinical vital sign’ could conceivably serve as a screening tool for identification of low CRF individuals at high risk of adverse outcomes, in whom a confirmatory gold standard CPET or other testing could be performed as clinically indicated. Deep ECG-\dot{V}O₂ may also represent an easily accessible marker to track patients' CRF trajectories over time, which may provide additive prognostic value. Applicants' approach further may be useful in identifying changes in CRF within a given individual using successive ECGs.

Importantly, use of deep learning to encode the 12-lead ECG for the purposes of \dot{V}O_{2 PEAK} estimation potentially offers mechanistic insights related to how CRF may manifest on the ECG. Inspection of median samples, a method of model interpretation utilizing visual inspection of exemplar waveforms corresponding to contrasting model predictions, showed clear associations between particularly high estimated \dot{V}O_{2 PEAK} with concordantly greater R and S wave amplitudes with more prominent ST segments and T waves. Collectively, these changes likely reflect electrical manifestations of structural adaptation to exercise training and CRF and may specifically relate to the process of physiologic hypertrophy associated with certain degrees of CRF. Future work is warranted to assess whether deep learned ECG representations can inform analyses (e.g., genomic, phenome-wide) with the potential to identify novel biological mechanisms and targets underlying human CRF.

In summary, Applicants demonstrated that automated estimation of \dot{V}O_{2 PEAK} from the resting 12-lead ECG is both feasible and accurate, with optimal model performance seen with our deep learning-based model, Deep ECG-\dot{V}O₂. In an ambulatory sample independent of model derivation, impaired CRF as predicted by Deep ECG-\dot{V}O₂ was robustly associated with incident disease, including heart failure and all-cause death. Automated \dot{V}O_{2 PEAK} estimation leveraging deep learning may enable rapid and scalable identification of individuals with impaired CRY who are at higher risk for cardiovascular events.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1.-21. (canceled)

22. A computer-implemented method to predict peak oxygen consumption, comprising:
generating, using a deep learning model, numerical representations of ECG waveform data recorded from an individual;
predicting, using a linear model, a peak oxygen consumption of the individual based on the numerical representations, the linear model being trained using cardiopulmonary exercise test (CPET)-derived peak oxygen consumption values; and
outputting the predicted peak oxygen consumption of the individual.

23. The computer-implemented method of claim 22, further comprising:
predicting a risk of one or more cardiovascular outcomes for the individual based on the predicted peak oxygen consumption of the individual.

24. The computer-implemented method of claim 22, wherein the deep learning model outputs the numerical representations as a 320-dimensional embedding.

25. The computer-implemented method of claim 22, wherein the ECG waveform data are measured while the individual is resting.

26. The computer-implemented method of claim 22, wherein the ECG waveform data are measured via a 12-lead ECG.

27. The computer-implemented method of claim 22, wherein the predicted peak oxygen consumption is an exercise peak oxygen consumption.

28. The computer-implemented method of claim 22, wherein the linear model is a penalized regression model trained to regress the peak oxygen consumption based on age, sex, body mass index, and CPET modality.

29. The computer-implemented method of claim 28, wherein the CPET modality is one of a bike, a treadmill, or a rowing ergometer.

30. The computer-implemented method of claim 28, wherein the penalized regression model uses a Ridge, an Elastic Net, a Least Absolute Shrinkage, or a Lasso penalty.

31. The computer-implemented method of claim 28, wherein the penalized regression model is trained to regress the peak oxygen consumption further based on measured intervals of the ECG waveform data.

32. The computer-implemented method of claim 31, wherein the measured intervals of the ECG waveform data include at least one of a ventricular rate, a PR interval, a QRS duration, or a QT interval.

33. A computer-implemented method, comprising,
generating, by a convolutional neural network, an embedding of a resting electrocardiogram (ECG) recorded from a subject;
estimating, by a penalized regression model, a peak oxygen consumption of the subject based on the embedding; and
outputting an estimate of a cardiorespiratory fitness of the subject based on the estimated peak oxygen consumption.

34. The computer-implemented method of claim 33, wherein the convolutional neural network is pre-trained independently of the penalized regression model.

35. The computer-implemented method of claim 33, wherein the penalized regression model is trained using a training set comprising data from a plurality of other subjects that had at least one ECG recorded within at least one year of a peak oxygen consumption measurement determined using cardiopulmonary exercise testing (CPET).

36. The computer-implemented method of claim 33, wherein the embedding is an at least 320-dimensional embedding indicating differences between ECGs from different individuals and similarities between ECGs from a same individual.

37. The computer-implemented method of claim 33, further comprising:
stratifying the subject with respect to at least one cardiovascular outcome based on the estimated peak oxygen consumption.

38. A system for determining peak oxygen consumption from electrocardiogram (ECG) waveform data, comprising:
a processor configured to execute instructions stored in a non-transitory memory to perform operations comprising:
generating, by a deep learning model, an embedding of a resting ECG recorded from a subject;
estimating, by a penalized regression model, a peak oxygen consumption of the subject based on the embedding, the penalized regression model trained based on resting ECG data from a plurality of other subjects and corresponding cardiopulmonary exercise test-derived peak oxygen consumption values;
predicting a risk of at least one cardiovascular outcome for the subject based on the estimated peak oxygen consumption; and
outputting the estimated peak oxygen consumption and the predicted risk.

39. The system of claim 38, wherein the penalized regression model uses at least one of a Ridge penalty, an Elastic Net penalty, a Least Absolute Shrinkage penalty, or a Lasso penalty.

40. The system of claim 38, wherein the embedding is an at least 320-dimensional embedding indicating differences between ECGs obtained from different individuals and similarities between ECGs obtained from a same individual.

41. The system of claim 38, wherein the at least one cardiovascular outcome includes a myocardial infarction
